(19) United States
(12) Patent Application Publication
(10) Pub. No.: US 2003/0012782 A1
(43) Pub. Date: Jan. 16, 2003

(54) Method and composition for altering a T cell mediated pathology

(76) Inventors: Daniel P. Gold, Del Mar, CA (US); Robert J. Shopes, San Diego, CA (US)

Correspondence Address: BROBECK, PHLEGER & HARRISON LLP, 12390 EL CAMINO REAL, SAN DIEGO, CA, 92130, US

(21) Appl. No.: 09/927,122

(22) Filed: Aug. 10, 2001

Related U.S. Application Data
(60) Provisional application No. 60/224,723, filed on Aug. 11, 2000.
(60) Provisional application No. 60/224,722, filed on Aug. 11, 2000.
(60) Provisional application No. 60/266,133, filed on Feb. 1, 2001.

(51) Int. Cl. A61K 39/395

(52) U.S. Cl. 424/133.1; 424/134.1

(57) ABSTRACT

The present invention provides a method for altering a T cell mediated pathology in a patient. This method comprises administering a composition comprising at least one and/or two chimeric proteins. Each chimeric protein comprises at least a portion of either the V_{α} or V_{β} region of a TCR from particular T cells from a patient having a T cell mediated pathology, and an immunoglobulin constant region. The genes encoding V_{α} and/or V_{β} regions and the genes encoding immunoglobulin constant regions are isolated and inserted into an expression vector. The chimeric proteins are produced by introducing the expression vectors into insect cell lines. The chimeric proteins are purified using antibody affinity columns, and then chemically conjugated to an immunogenic carrier, keyhole-limpet hemocyanin (KLH). Since the conjugates comprises chimeric proteins made specifically from particular T cells from a patient having T cell mediated pathology, when it is administered to such a patient, with or without a cytokine, such as granulocyte-macrophage-CSF, or a chemokine, it can induce immune responses to alter such a T cell mediated pathology.

### RELATED APPLICATIONS

This application claims priority to the U.S. Provisional Application No. 60/224,723, entitled “Method for Producing an Idiotypic Vaccine,” the U.S. Provisional Application No. 60/224,722 entitled “Expression Vectors for Production of Recombinant Immunoglobulin” and the U.S. Provisional Application No. 60/266,133 entitled “Method and Composition for Altering a T Cell Mediated Pathology.”

### FIELD OF THE INVENTION

This invention relates generally to the field of immunology and immunotherapy.

More specifically, this invention relates to methods and compositions for altering T cell mediated pathologies, such as T cell malignancies and/or autoimmune diseases.

### BACKGROUND OF THE INVENTION

This invention relates generally to the field of immunology and immunotherapy.

More specifically, this invention relates to methods and compositions for altering T cell mediated pathologies, such as T cell malignancies and/or autoimmune diseases.

The immune system produces both antibody-mediated and cell-mediated responses. Each type of immune response is regulated by a type of lymphocytes, B cells (for antibody-mediated response) and T cells (for cell-mediated response). T cells bind to certain foreign proteins (antigens) when portions of the antigen associate with a major histocompatibility complex (“MHC”), typically through an antigen presenting cell (“APC”) in which the antigen is digested into fragments and presented on the surface of the APC bound to its MHC.

T cell lymphoma is a T cell mediated pathology that results from inappropriate replication of T cells that eventually results in an impaired functioning of the immune system. T cell lymphomas are difficult to treat effectively with the currently available medications and additional therapeutic strategies would be welcome additions to the physician's arsenal.

Other T cell mediated pathologies include a growing number of human diseases that have been classified as autoimmune diseases, where the host's own immune system attacks the host's own tissue. T cells are one of the primary regulators of the immune system and directly or indirectly affect such autoimmune pathologies. Examples of such autoimmune diseases are rheumatoid arthritis (RA), myasthenia gravis (MG), multiple sclerosis (MS), systemic lupus erythematosus (SLE), autoimmune thyroiditis (Hashimoto's thyroiditis), Graves' disease, inflammatory bowel disease, autoimmune uveoretinitis, polymyositis and certain types of diabetes. The present treatment for these autoimmune diseases do not cure the disease but, instead, only treat the symptoms.

It is now known that these and other autoimmune diseases involve the action of T helper cells stimulated by the binding of their T cell receptor (TCR) to an MHC/autoantigen (or nonautoantigen) complex. It has been proposed that possible treatment of these autoimmune diseases may be accomplished by disrupting the interaction between the MHC/antigen complex and the TCR.

The TCR found on the surface of most mature T lymphocytes is an heterodimeric integral membrane protein most commonly comprising α and β chains. The overall three dimensional structure of the TCR is similar to that of cell surface associated immunoglobulin (Ig) on B cells, in that each α and β chain contains an amino terminal variable (V) region responsible for antigen recognition and a carboxy terminal constant (C) region which is critical in signaling the recognition event to the inside of the cell. The β chain also contains additional amino acids that are encoded by the diversity (D) and joining (J) gene segments. These V, D, J and C gene segments are located at distinct and separate locations on the chromosome. The V_{β}, D_{β}, J_{β}, and C_{β} gene segments are recombined to create the functional transcription unit that encodes the β chain. The functional α chain transcription unit is formed by recombination of V_{α}, J_{α}, and C_{α} segments. The unique recombination of the V_{α}, J_{α}, and C_{α} gene segments, plus V_{β}, D_{β}, J_{β}, and C_{β} gene segments in a clone of lymphocytes gives rise to an unique protein determinant, the idiotype (Id), found only in that particular lymphocyte.

Important differences exist which distinguish TCR molecules from immunoglobulin molecules. Immunoglobulins generally recognize their ligand in solution phase, either as B cell antigen receptors, or as secreted molecules, while TCRs only recognize ligands as peptide fragments from protein antigens in association with MHC class I and class II molecules residing on the cell surface of other cells. In addition, unlike immunoglobulins, no alternate splicing of TCR mRNA occurs to yield secreted TCR chains, and thus TCR molecules exist only as integral membrane proteins on the surface of T cells. Finally, when T cells are activated following antigen recognition, no hypermutation of the TCR variable region gene segments occur, in contrast to V-gene segments of immunoglobulin in B cells, which do undergo somatic mutations and increase their affinity for their ligands.

Based on extensive studies of autoimmune disease, it is now well documented that T cells and their expressed TCR chains can be the targets of immunoregulation by other T cells (Vandenbark et al, “Human TCR as antigen: Homologies and potentially cross-reactive HLA-DR-2 restricted epitopes with the AV and BV CDR2 loops,” Critical Reviews in Immunol., 20:57-83, 2000). It is currently thought that peptide fragments of TCR chains are enzymatically processed and subsequently presented on the cell surface of activated T cells in association with both MHC class I and class II molecules. MHC class II molecules are only found on T cells following activation, and recent evidence suggests that TCR peptide presentation occurs via a subset of MHC class I molecules whose expression is also upregulated following activation (Ware et al., Immunity 2:177-184, 1995; Jiang et al., Immunity 2:185-94, 1995). These MHC/TCR-peptide complexes induce auto-regulatory immunity in the form of MHC class I restricted CD8⁺ and MHC class II restricted CD4⁺ effector T cells. Reports of the immunoregulatory properties of selected TCR peptide vaccines in the setting of T cell mediated autoimmune disease have been made (Gold et al., Critical Reviews Immunol., 17:507-10, 1997). Moreover, because whole TCR chains are clonally distributed on T cells (as is immunoglobulin on clones of B cells), intact, functional TCRs are candidates as potential tumor specific antigens for T cell lymphomas. Among the potential disadvantages of using the intact TCR as an antigen is complications which may result from generating an immune response to the constant regions of the α and β chains.

Immunoregulatory cell mediated responses specific for TCR chains appear to be directed against germline encoded determinants in the variable regions of the TCR chain. Since TCR chains are not prone to mutations like Ig molecules, the therapeutic use of generic TCR chains was suggested by experimental evidence in animal studies. Vaccination with secreted TCR β chains made in bacteria (Kumar, J. Immunol. 159:5150-56, 1997), with vaccinia virus constructs expressing TCR β chains (Chundru, J. Immunol. 156:4940-45, 1996), or with “naked” DNA constructs encoding single TCR β chains (Waisman, Nat. Med. 2:899-905, 1996) all resulted in the induction of regulatory T cell responses directed against the immunizing TCR β chains.

The use of peptide vaccines based on unique TCR motifs that have a defined chemical nature as therapeutic agents for treating T cell-mediated pathologies has been tested. Some researchers have shown that framework/CDR region derived peptides of TCR chains could prevent autoimmune disease onset in animals if treated with peptides that were derived from TCR chains that were over-expressed in the particular disease setting. This demonstrates that T cells process their own endogenous TCR chains and re-present them to other T cells in a manner such that the ensuing T cell-T cell interaction could function to regulate a pathogenic cell. However, because TCR peptide recognition requires that the TCR chain be processed into peptide fragments that are presented on the target T cell surface in association with MHC molecules, it is extremely difficult to predict in advance which peptide from a particular TCR will be effective in a clinical setting. This is illustrated by a survey of studies in inbred rodents demonstrating MHC class II restricted responses to TCR peptides (Gold et al., Critical Reviews Immunol., 17:507-10, 1997, supra). Thus, like most proteins, both the V_{β} sequence and the MHC determine which epitope will be stimulatory for T cells, and no apparent rule predicts where in the V_{β} amino acid sequence the relevant TCR peptide will reside. This challenge is compounded by the extensive degree of MHC polymorphism that exists in human populations.

Production of a TCR idiotype to modulate the T cell mediated immune response is difficult because the TCR is an integral membrane protein and is not normally synthesized and secreted as a soluble protein. Thus, it is not practical to routinely purify a sufficient amount of TCR from pathogenic cells, tumor cells for example, for use in therapeutic applications.

Attempts to produce a large amount of a secreted form of the heterodimeric TCR have been made using insect cells. These secreted molecules contained the entire V_{α}J_{α}C_{α}/V_{β}D_{β}J_{β}C_{β} extracellular domains and folded correctly based on their detection with conformationally-specific anti-TCR V_{β} antibodies. However, the production level of the secreted molecules can be low. Other investigators have used various methodologies to produce recombinant TCR chains, all with the stated intent of making a soluble heterodimeric structure that would produce a functional TCR that would recognize its cognate partner, MHC+peptide.

Efforts to produce soluble portions of the TCR for therapeutic purposes using several different methodologies have encountered other significant problems. These and related efforts include: (1) production of single chain Fv constructs in E. coli that can be purified from inclusion bodies and subsequently refolded (Kurucz et al, Proc. Natl. Acad. Sci., 90:3830-34, 1994); (2) production of recombinant TCR molecules in bacteria (Kumar et al., J. Immunol. 159:5150-56, 1997; Offner et al., J Immunol. 161:2178-86, 1998; Novotny et al., Proc. Natl. Acad. Sci., 88:8646-50, 1991), (3) production of chimeric molecules made by fusions between TCR variable regions plus all or almost all of the TCR constant regions and Ig constant regions which can be secreted from mammalian cells (Gregoire et al., Proc. Natl. Acad. Sci. 88:8077-81, 1991; Weber et al., Nature 356:793-96, 1992; Eilat, Proc. Natl. Acad. Sci., 89:6871-75, 1992), (4) expression of the extracytoplasmic domains of the TCR on the surface of mammalian cells via a phosphatidyl inositol glycan linkage which can then be cleaved through the action of a phosphatidyl inositol-specific phospholipase C (Lin et al., Science 249:677-79, 1990; Chung et al., Proc. Natl. Acad. Sci. 91:12654-58, 1994; Okada et al. J. Immunol., 159:5516-27, 1997), and (5) expression of single or heterodimeric complete TCR chains as secreted proteins in insect cells (Kappler et al., Proc. NatL. Acad. Sci., 91:8462-66, 1994). The drawbacks associated with each of the methods that utilize bacteria include certainly solubility and possible endotoxin contamination issues which detract from the ease and high yield potential of proteins made in bacteria. For proteins made in mammalian cells, concerns about viral load arise. These concerns, compounded with the difficulties in purifying phospholipid-associated proteins make this approach even less attractive for clinical settings.

Additional difficulties are encountered by antibodies produced in E. coli that are not generally useful for therapeutic applications in spite of their usefulness in the identification of genes encoding desired binding specificities. Typically, only the antibody's antigen binding fragments, Fab or Fv, are secreted in bacteria (see, e.g., Kurucz et al., Proc. Natl. Acad. Sci., 90:3830-34, 1994). In the rare instance when a whole chain tetrameric IgG has been produced in E. coli, the antibodies are improperly glycosylated. Without proper glycosylation, antibodies will not trigger the cytolytic activities of antibody-directed cellular cytotoxicity (ADCC) and complement activation that make passive immunotherapy so powerful. Mammalian expression systems, on the other hand, produce glycosolated antibody. However, recent modifications in the CBER division of he FDA's “Points to Consider” clearly signal their concerns about viral loads associated with monoclonal antibodies produced in mammalian cell expression systems. Moreover, it is expected that any engineered antibody produced in a mammalian expression system will be quite expensive ($1500-$5000 per dose).

The baculovirus expression system is an excellent alternative to antibody production in E. coli and mammalian cells. High yield production (1-100 mg/L) of biologically active proteins in eukaryotic cells is possible using the baculovirus system (Haseman et al., Proc. Natl. Acad. Sci. 87:3942-46, 1990). The baculovirus/insect cell system circumvents the solubility problems often encountered when recombinant proteins are overexpressed in prokaryotes. In addition, insect cells contain the post-translational modification machinery responsible for correct folding, disulfide formation, glycosylation, β-hydroxylation, fatty acid acylation, prenylation, phosphorylation and amidation present of eukaryotic cells. The production of a functional, glycosylated monoclonal antibody recognizing human colorectal carcinoma cells from a baculovirus expression system has been recently demonstrated (Nesbit, J. Immunol. Methods, 151:201-208, 1992). This baculovirus-produced antibody was shown to mediate ADCC; in contrast, antibodies produced in bacteria are not glycosylated and therefore have no detectable ADCC activity.

In some instances, utilization of the baculovirus system for the expression of biologically active proteins has been hampered by the inability to efficiently solubilize recombinant proteins without excessive proteolytic degradation. In order to circumvent solubility and proteolysis problems encountered with the expression of recombinant proteins in insect cells, baculovirus transfer vectors were developed for the efficient secretion of biologically active proteins. These vectors that facilitate the secretion of recombinant proteins from host insect cells are constructed by inserting functional secretory leader sequences downstream of the polyhedrin promoter. In-frame insertion of cDNA sequences resulted in the synthesis of proteins containing a heterologous signal sequence which directed the recombinant protein to the secretory pathway. Human and insect leader sequences were both tested to maximize secretion of heterologous proteins from insect cells. The human placental alkaline phosphatase signal sequence (MLGPCMLLLLLLLGLRLQLSLG (SEQ ID NO:1); DNA sequence: ATG GTG GGA CCC TGC ATG CTG CTG CTG CTG CTG CTG CTA GGC CTG AGG CTA CAG CTC TCC CTG GGC (SEQ ID NO:2)) and the honeybee melittin signal sequence (MKFLVNVALVFMVVYISYIYA (SEQ ID NO:3); DNA sequence: ATG AAA TTC TTA GTC AAC GTT GCA CTA GTT TTT ATG GTC GTG TAC ATT TCT TAC ATC TAT GCG (SEQ ID NO:4)) have both proved useful for the secretion of numerous bacterial and human proteins (Mroczkowski et al., J. Biol. Chem. 269:13522-28, 1994 and Tessier et al., Gene 98:177-83, 1991).

Using baculovirus expression systems, McKeever et al. (J.Exp. Med 184:1755-68, 1996) were able to produce a chimeric protein composed extracellular domains of the TCR chains (V_{α}-C_{α} and V_{β}-C_{β}) linked to the hinge, CH₂ and CH₃ domains of the mouse IgG₁ heavy chain. The resulting solubilized TCR-IgG₁ chimeric proteins from a T cell clone specific for a pancreatic β-cell antigen were used to immunize female nonobese diabetic (NOD) mice. After mating, the offspring of these mice were analyzed to determine the effect of maternally transferred anti-TCR antibodies on the diabetogenic activity. These studies demonstrated that when administered in a soluble form, the variable region of the α or β chains of the TCR can be immunogenic in mice of the strain from which it was derived. These studies also demonstrated that immunization with soluble TCR-IgG₁ could stimulate the production of antibodies recognizing native clonotypic epitopes. McKeever et al. clearly demonstrated that using a relatively simple expression and purification strategy, it is possible to produce a soluble TCR-IgG₁ protein containing all of the α and β chains in which the TCR portion possesses clonotypic determinations that are immunogenic and cross-reactive with those found in the functionally active cell-surface form of the TCR.

Several other investigators have also expressed TCR molecules as molecular fusions to Ig chains in various ways. However, all these studies used the entire TCR structure, i.e., both the variable and constant regions, to fuse to various parts of an Ig backbone, and all had stated intent of reproducing the recognition function of the TCR variable regions. It was also demonstrated that various forms of TCR chains could be used as vaccines in animal studies for the prevent/treatment of autoimmune disease. For example, Okada et al (J. Immunol., 159:5516-27, 1997) have shown that soluble TCR chains made from mammalian cells could be used to treat a T cell tumor in mice. As discussed above, however, the production of these TCR molecules was difficult, and the production level was low.

Additionally, the use of TCR proteins is predicted to have therapeutic value in veterinary applications (see International Patent Application No. PCT/US99/17309, WO 00/06733, filed Jul. 29, 1999).

### SUMMARY OF THE INVENTION

The present invention provides a method for altering a T cell mediated pathology in a patient. This method includes administering a composition that contains at least one chimeric protein having at least a portion of a V_{β} or V_{α} chain of a TCR and at least a portion of an immunoglobulin constant region. In other preferred embodiments, the chimeric proteins present may comprise at least a portion of a V_{β} or V_{α} chain of a TCR, plus a linker region, plus at least a portion of an immunoglobulin constant region. In further preferred embodiments, the portion of the TCR constant region in the chimeric protein is from about 3 to about 30 contiguous amino acid residues. The linker region may comprise a portion of the TCR constant region. In still other preferred embodiments, the portion of the constant region in the chimeric protein comprises the amino acid residues up to and including the first cysteine residue of the immunoglobulin fold of the constant region. The V_{β} or V_{α} chain used in this composition is associated with a particular TCR from a T cell from the patient having a T cell mediated pathology. After administering such a composition into a patient, the T cell mediated pathology in the patient is altered.

The present invention also provides a method for altering a T cell mediated pathology in a patient by administering a composition containing two different chimeric proteins. Each chimeric protein has at least a portion of a V_{β} or V_{α} chain of a TCR linked to at least a portion of an immunoglobulin constant region. In other preferred embodiments, the chimeric proteins present may comprise at least a portion of a V_{β} or V_{α} chain of a TCR, plus a linker region, plus at least a portion of an immunoglobulin constant region. In further preferred embodiments, the portion of the TCR constant region in the chimeric protein is about 3, 4, 5, 6, 7, 8, 9, 10, 14, 18, 22, 26, or 30 contiguous amino acid residues. The linker region may comprise a portion of the TCR constant region. In still other preferred embodiments, the portion of the constant region in the chimeric protein comprises some or all of those amino acid residues to the amino-terminal side of the first cysteine residue of the immunoglobulin fold of the constant region. The V_{β} and/or V_{α} chains that are part of the chimeric protein are associated with a particular TCR from a T cell of the patient having a T cell mediated pathology.

Specific TCR proteins containing patient-derived unique V_{α} and/or V_{β} chains can be developed as therapeutic compositions. Suspected self-antigens can be used to selectively stimulate and expand T cells involved in autoimmune T cell pathologies, such as rheumatoid arthritis (RA), myasthenia gravis (MG), multiple sclerosis (MS), systemic lupus erythematosus (SLE), autoimmune thyroiditis (Hashimoto's thyroiditis), Graves' disease, inflammatory bowel disease, autoimmune uveoretinitis, polymyositis and certain types of diabetes. Alternatively, T cells that are isolated from a tissue undergoing autoimmune attack can be selectively expanded by use of T cell growth factors, i.e., IL-2, IL-4. T cells associated with multiple sclerosis have been characterized in this manner by Wilson et al. (J. Neuroimmunol. 76:15-28, 1997) which hereby is incorporated by reference in its entirety, including any drawings, figures, and tables. T cells which are associated with other T cell mediated pathologies may be characterized by an adaptation of the methods described in Wilson et al. Following the purification of a small number of pathogenic T cells, the variable portion of T cell receptors expressed by these cells may be cloned via PCR using the methods described in the invention. Once cloned, the V_{α} and/or V_{β} portions of the receptors specifically involved in the T cell pathology can be used to make chimeric proteins which can be expressed in a baculovirus system as described herein.

The immunoglobulin constant regions used in the above compositions and chimeric proteins can be a portion of a protein selected from the group consisting of IgG₁, IgG₂, IgG₃, IgG₄, IgA, IgA₁, IgA₂, IgM, IgD, IgE heavy chains, κ and λ light chains. In some of the embodiments, the chimeric protein only contains either the V_{β} or V_{α} chain of a TCR with an immunoglobulin constant region. Examples of chimeric proteins include V_{β}-IgG_{γ1}, V_{α}-κ, or V_{α}-λ, or V_{α}-IgG_{γ1}, V_{β}-κ, or V_{α}-λ. In another embodiments, the composition contains two chimeric proteins that each respectively contains a V_{β} and V_{α} chain with an immunoglobulin constant regions. Examples include V_{β}-IgG_{γ1} and V_{α}-κ, or V_{β}-IgG_{γ1}, and V_{α}-λ, or V_{α}-IgG_{γ1}, and V_{α}-κ, or V_{α}-IgG_{γ1} and V_{β}-λ. In other preferred embodiments, the chimeric proteins may comprise at least a portion of a V_{β} or V_{α} chain of a TCR, plus a linker chain, plus at least a portion of an immunoglobulin constant region. In certain preferred embodiments, the portion of the TCR constant region in the chimeric protein is about 3, 4, 5, 6, 7, 8, 9, 10, 14, 18, 22, 26, or 30 contiguous amino acid residues. In still other preferred embodiments, the portion of the constant region in the chimeric protein comprises some or all of those amino acid residues to the amino-terminal side of the first cysteine residue of the immunoglobulin fold. Examples include V_{β}-C_{β}-IgG_{γ1} and V_{α}-C_{α}-κ, or V_{β}-C_{β}-IgG_{γ1} and V_{α}-C_{α}-λ, or V_{α}-C_{α}-IgG_{γ1} and V_{β}-C_{β}-κ, or V_{α}-C_{α}-IgG_{γ1} and V_{β}-C_{β}-λ.

The present invention also provides a method for producing chimeric proteins using recombinant DNA technology and an expression system. This method includes the following steps: (a) isolating genes encoding V_{β} or V_{α} chains of a TCR from T cells of a patient having a T cell mediated pathology, (b) inserting the gene encoding the V_{β} or V_{α} chain of the TCR and the gene encoding the immunoglobulin constant region into an expression vector to allow the expression of a chimeric protein, (c) producing the chimeric protein by introducing the expression vector into insect cell lines and allowing its expression, and (d) isolating the chimeric protein. The method for producing chimeric proteins further includes a step of inserting a gene encoding either V_{β} or V_{α} chain of the TCR and genes encoding a second immunoglobulin constant region into the expression vector to allow the expression of the second chimeric protein. The isolated genes encoding the V_{β} or V_{α} chains of a TCR may additionally comprise a portion of the TCR's constant region of about thirty amino acids or less.

The present invention further provides a composition for altering a T cell mediated pathology in a patient. This composition contains at least one chimeric protein having at least a portion of a V_{β} or V_{α} chain of a TCR and at least a portion of an immunoglobulin constant region. In other preferred embodiments, the chimeric proteins may comprise at least a portion of a V_{β} or V_{α} chain of a TCR, plus a portion of that TCR chain's constant region of about thirty amino acid residues or less, and at least a portion of an immunoglobulin constant region. In further preferred embodiments, the portion of the TCR constant region in the chimeric protein is about 3, 4, 5, 6, 7, 8, 9, 10, 14, 18, 22, 26, or 30 contiguous amino acid residues. In still other preferred embodiments, the portion of the TCR constant region in the chimeric protein comprises some or all of those amino acid residues to the amino-terminal side of the first cysteine residue of the immunoglobulin fold of the constant region. The V_{β} or V_{α} chain that is part of the chimeric protein is associated with a particular TCR from a T cell of a patient having a T cell mediated pathology. The composition further contains a second chimeric protein having at least a portion of a V_{β} or V_{α} chain of a TCR and at least a portion of a second immunoglobulin constant region. In other preferred embodiments, the chimeric proteins may comprise at least a portion of a V_{β} or V_{α} chain of a TCR, plus a linker region, plus at least a portion of an immunoglobulin constant region. In further preferred embodiments, the linker region may comprise a portion of the TCR constant region which is about 3, 4, 5, 6, 7, 8, 9, 10, 14, 18, 22, 26, or 30 contiguous amino acid residues. In still other preferred embodiments, the portion of the constant region in the chimeric protein comprises some or all of some or all of those amino acid residues to the amino-terminal side of the first cysteine residue of the immunoglobulin fold of the constant region. The V_{β} or V_{α} chain that is part of the second chimeric protein is associated with a particular TCR from a T cell of a patient having a T cell mediated pathology.

In one of the embodiments of the invention, the composition contains two chimeric proteins, the first one comprising the entire V_{β} region and a human constant region of an immunoglobulin IgG_{γ1} (TCR V_{β}-IgG_{γ1}), the second one comprising the entire V_{α} and a human κ or λ constant (TCR V_{α}-κ or TCR V_{α}-λ). In other preferred embodiments, either or both of the chimeric proteins may comprise at least a portion of a V_{β} or V_{α} chain of a TCR, plus a linker region, plus at least a portion of an immunoglobulin constant region. In further preferred embodiments, the linker region in either or both of the chimeric proteins may comprise a portion of a TCR constant region which is about 3, 4, 5, 6, 7, 8, 9, 10, 14, 18, 22, 26, or 30 contiguous amino acid residues.

In another embodiment of the invention, the composition contains two chimeric proteins, the first one comprised the entire V_{β} region plus a portion of the TCR β chain constant region (C_{β}) of thirty amino acid residues or less and a human constant region of an immunoglobulin IgG_{γ1} (TCR V_{β}C_{β(1-30)}-IgG_{γ1}). In preferred embodiments, the portion of the C_{β} used is about 3, 4, 5, 6, 7, 8, 9, 10, 14, 18, 22, 26, or 30 contiguous amino acid residues. The second chimeric protein comprises the entire V_{α} plus a portion of the TCR α chain constant region (C_{α}) of thirty amino acid residues or less and a human K constant region (TCR V_{α}C_{α(1-30)}-κ). In preferred embodiments, the portion of the C_{α} used is about 3, 4, 5, 6, 7, 8, 9, 10, 14, 18, 22, 26, or 30 contiguous amino acid residues.

In another embodiment of the invention, the composition contains a single chimeric protein containing either V_{β} or V_{α} chain from a particular TCR from a T cell of a patient and an immunoglobulin constant region. Examples include chimeric proteins V_{β}-IgG_{γ1}, V_{α}-κ, or V_{α}-λ, or V_{α}-IgG_{γ1}, V_{β}-κ, or V_{β}-λ. In other preferred embodiments, the chimeric proteins may comprise at least a portion of a V_{β} or V_{α} chain of a TCR, plus a linker region, plus at least a portion of an immunoglobulin constant region. In further preferred embodiments, the linker region in either or both of the chimeric proteins may comprise a portion of a TCR constant region which is about 3, 4, 5, 6, 7, 8, 9, 10, 14, 18, 22, 26, or 30 contiguous amino acid residues.

In one of the embodiments of the invention, the expression vector used to express the chimeric proteins is a baculovirus vector. The vector contains two expression cassettes each having a promoter, a secretory signal sequence and a chimeric protein. One expression cassette contains the baculovirus AcNPV p10 promotor linked to the honey bee melittin signal sequence. The other expression cassette has the polyhedrin promotor linked to a human placental alkaline phosphatase signal sequence. In addition to the listed promoters and signal sequences other promoters and signal sequences known to those skilled in the art could be substituted. In preferred embodiments, the endogenous secretory sequences associated with the immunoglobulin genes derived from a given patient are used. The genes encoding the V_{α} or V_{β} chains of the TCR, the linker region, and the immunoglobulin constant region are inserted, separately and/or together, into the above expression cassette of the baculovirus vector allowing expression of one or two chimeric proteins. In a preferred embodiment, the constant region of the immunoglobulin heavy chain, such as IgG₁, together with either the V_{β} or V_{α} chain, is controlled by the polyhedrin promotor.

Chimeric proteins produced are purified using affinity columns with antibodies of anti-immunoglobulin or Ig-binding proteins, such as Protein A for the constant region of an immunoglobulin heavy chain, Protein L for variable regions of an immunoglobulin Kappa light chain, and/or any other proteins that bind to an immunoglobulin binding domain.

The present invention also contemplates covalently coupling the chimeric proteins to carrier proteins such as keyhole limpet hemocyanin (KLH). The composition of the present invention may also be administered into a patient together with a cytokine, such as granulocyte-macrophage-CSF (GM-CSF), or a chemokine, such as a monocyte chemotactic protein 3 (MCP 3). Because the present composition of the present invention containing chimeric protein(s) is specifically related to a particular TCR from T cells of a patient having a T cell mediated pathology, administration of this composition induces an immune response against the disease specific idiotype in which particular V_{α} or V_{β} segments are involved. Similar responses against T cells associated with autoimmune diseases involving T cells that use a restricted repertoire of TCR V-region segments, such as V_{α} or V_{β} segments. Thus, the administration of the composition of the present invention alters a T cell mediated pathology and/or autoimmune diseases in a patient. The administration routes for the invented composition include, but are not limited to, oral delivery, delivery via inhalation, injection, transdermal delivery, and the like.

All U.S. patents and applications; foreign patents and applications; scientific articles; books; and publications mentioned herein are hereby incorporated by reference in their entirety, including any drawings, figures and tables, as though set forth in full.

### BRIEF DESCRIPTION OF THE DRAWINGS

FIG. 1: A general scheme for producing a composition comprising chimeric proteins for V_{α} or V_{β} chain of a particular TCR from T cells from a patient having T cell mediated pathology.

FIG. 2: Plasmid map of a baculovirus expression vector p2Bac with multiple cloning sites.

FIG. 3: DNA sequence of baculovirus expression vector p2Bac (SEQ ID NO:5). The sequence is depicted from 5′ to 3′. The p2Bac vector contains the AcNPV polyhedrin gene promoter (nucleotides 1 to 120 of the GenBank accession number X06637 (SEQ ID NO:44)) and the AcMNPV p10 promoter (nucleotides 8 to 237 of GenBank accession number A28889 (SEQ ID NO:45)).

FIG. 4: DNA sequence of the plasmid pTRABac/9F12. This plasmid contains the genes for the heavy and light (κ) chains expressed by the stable human cell-line 9F12 (SEQ ID NO:41). The 9F12 cell line produces a human IgG1/κ antibody specific for tetanus toxoid. The underlined regions represent sequences encoding mature 9F12 IgG₁ (TTTACCC . . . ) and kappa (ATCGACA . . . ) chains, respectively. The sequence is depicted from 5′ to 3′.

FIG. 5a: Plasmid map of recombinant baculovirus expression vector pTRABacHuLC_{κ}HC_{γ1} using IgG_{γ1} and κ constant regions.

FIG. 5b: Plasmid map of recombinant baculovirus expression vector pTRABacHuLC_{λ}HC_{γ1} with IgG_{γ1} and λ constant regions.

FIG. 6A: DNA sequence of pTRABacHuLC_{κ}HC_{γ1} (SEQ ID NO:6). The sequence is depicted from 5′ to 3′.

FIG. 6B: DNA sequence of pTRABacHuLC_{λ}HC_{γ1} (SEQ ID NO:7). The sequence is depicted from 5′ to 3′.

FIG. 6C: DNA sequence of pTRABacHuLC_{κ}HC_{γ1} following modification utilizing the kappa stuff primers (SEQ ID NO:42). The sequence is depicted from 5′ to 3′.

FIG. 6D: DNA sequence of pTRABacHuLC_{λ}HC_{γ1} following modification utilizing the lambda stuff primers (SEQ ID NO:43). The sequence is depicted from 5′ to 3′.

FIG. 7: Representative protein sequences for TCR alpha chains and beta chains. The constant region of both sequences is underlined, and the first cysteine residue of the immunoglobulin fold is marked with a double underline (SEQ ID NO:24 and SEQ ID NO:25).

FIG. 8a: Plasmid map of recombinant baculovirus expression vector pTRABacHuLC_{κ}HC_{γ1} using IgG_{γ1} and κ constant regions showing insertion sites for V_{α} and V_{β} regions.

FIG. 8b: Plasmid map of recombinant baculovirus expression vector pTRABacHuLC_{λ}HC_{γ1} with IgG_{γ1} and λ constant regions showing insertion sites for V_{α} and V_{β} regions.

FIG. 9: Treatment of tumor bearing mice with a TCR V_{β}-Ig Chimeric Protein Formulation.

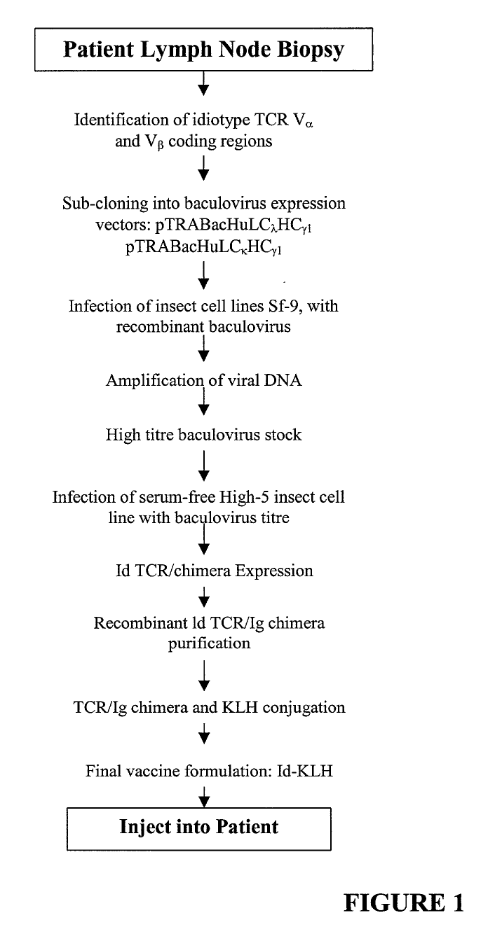

### DETAILED DESCRIPTION OF THE PREFERRED EMBODIMENTS

In the instant invention, the unique specificity of the immune system has been adapted to treat T cell malignancies and pathologies. In the instant invention, the DNA sequence encoding the variable region of the T cell receptor was cloned using primers derived from the 5′ end of each unique TCR V gene family and a TCR constant region primer. Typically, this process uses one of several suitable cloning techniques such as PCR. These TCR constant region primers, one for the α chain and one for the β chain, were used to clone the variable regions to produce a chimeric protein consisting of V_{α} or V_{β} plus an IgG₁ constant region. Additionally, one of skill in the art would be able to select several different primers that could be used equivalently in this system to produce equivalent results to amplify any pair of antibody variable regions for easy subcloning. Alternatively, techniques such as 5′ RACE may be used to clone the variable regions of the TCR chains as a step in producing a chimeric protein.

This chimeric protein was produced in insect cells using a baculovirus vector. These chimeric proteins produced without all of the entire C_{α} or C_{β} domain are distinguishable from chimeric TCR/Ig molecules that have the entire α or β chain present. For example, these chimeric proteins are not recognized by some conformationally specific antibodies directed against the TCR variable region, such as the anti-mouse V_{α}6 (clone RR4-7) and the anti-mouse V_{β}12 (clone MR 11-1) (PharMingen, San Diego, Calif.). These unique chimeric proteins are predicted to be superior antigens for vaccinations since the normal determinants that might complicate therapy will not be present to provoke an autoimmune reaction. Additionally, prior attempts at producing soluble TCR chimeric proteins were directed at proteins that adopted the native confirmation of the TCR in order to allow binding to an appropriate peptide/MHC complex.

The present invention fills the great demand for an effective treatment for T cell mediated pathologies and autoimmune diseases. The inventions take advantage of the unique cell surface antigens (idiotypes) present on the surface of T cells involved in T cell pathologies. To do so, there is a need to prepare vaccines in a patient-specific manner. Such vaccines provide exquisite selectivity by being tailored to the markers unique to the pathogenic T cells found in a given patent.

To tailor the present invention to a particular patient first requires identification and isolation of the unique antigens, and then the means of producing those antigens. Producing these antigens may be accomplished in a number of different ways available to one of skill in the art. For example, a recently developed method that is adapted to the needs of the instant invention uses a novel baculovirus/insect cell expression system and was recently developed for the efficient production of functional antibodies for immunotherapy (see U.S. Provisional Application Serial No. 60/244,722, entitled “Expression Vectors for Production of Recombinant Immunoglobulin”). This baculovirus expression vector was designed such that only two custom gene specific primers were needed to amplify any pair of antibody variable regions for easy subcloning and expression as chimeric proteins with the human kappa light chain or IgG₁ heavy chain. The incorporation of heterologous secretory signal sequences, which directed the heavy and light chains to the secretory pathway, were incorporated for the expression of large amounts of active immunoglobulin from insect cells. This vector should be useful for the expression of any kappa light chain variable region (V_{L}) in frame with human kappa constant region and secreted via the human placental alkaline phosphatase secretory signal sequence, and any heavy chain variable region (V_{H}) in frame with the human IgG1 constant domain preceded by the honey bee melittin secretory signal sequence. Any monoclonal antibody, mouse or human, either from a monoclonal cell line or identified by phage display cloning, could be easily expressed as whole human IgG₁/κ or IgG₁/κ in this vector after two simple subcloning steps.

In the instant invention, this baculovirus expression vector was designed such that only two custom gene specific primers were needed to amplify any pair of TCR variable regions for easy subcloning and expression as a fused V_{α} chain, linker, and IgG₁ heavy chain. The incorporation of heterologous secretory signal sequences, which directed the V_{α} and V_{β} chains to the secretory pathway, allows the expression of large amounts of chimeric protein from insect cells. This vector should be useful for the expression of any TCR α chain variable region (V_{α}) in frame with an Ig kappa constant region and secreted via the human placental alkaline phosphatase secretory signal sequence, and any TCR β chain variable region (V_{β}) in frame with an IgG₁ constant region and secreted via the honey bee melittin secretory signal sequence, or vice versa.

Expression of recombinant proteins using the baculovirus system allows the production of large quantities of biologically active proteins without many of the drawbacks associated with proteins made in bacteria, and also avoids the complications of using mammalian cells. For example, using a commercially available baculovirus single promoter vector (pVL1392) or a dual promoter vector (pAcUW51) (both can be obtained commercially from BD-Pharmingen), secreted TCR rat chains were produced, either as monomeric β chains or as heterodimeric αβ pairs using serum free medium. The presence of these chains can be detected in an ELISA using an anti-C_{β} constant region capture antibody (R73, Pharmingen) and an anti-V_{β} detection antibody (R78; anti-rat V_{β}8.2 or HIS 42; anti-rat V_{β}16, Pharmingen). Production levels were determined to be approximately 300 ng/ml. The TCR chains were made and secreted in native conformation, and the α and β chains formed stable disulfide linked heterodimers (Gold et al., 1997, supra).

Soluble human TCR fragments containing specific epitopes of the particular V segments can be produced in insect host cells via genetic engineering. These soluble recombinant TCR proteins containing particular V_{α} and/or V_{β} chains derived from a patient, can be used as a therapeutic composition. When it is administered into the patient, it would specifically induce, in vivo, a cell mediated immune response for altering the T cell mediated pathology.

This technology has also been applied towards the rapid identification and cloning of patient-specific V_{H} and V_{L} genes expressed by B cell lymphoma, then expressing these as recombinant I_{G1}/κ or λ molecules in insect cells (see U.S. Provisional Application Serial No. 60/224,723 entitled “Method for Producing Idiotype-Vaccine”). Molecules produced by this method were formulated and used to induce anti-idiotypic cell-mediated immunity against lymphomas in a patient-specific fashion.

The term “altering” or “alters” refers to the ability of a compound of the invention to modulate a T cell mediated pathology. A compound which alters a T cell pathology may do so by a number of potential mechanisms including raising an antibody response directed at the compound which in turn destroys cells of the T cell pathology, induces apoptosis in the cells of the T cell pathology, inhibits the further growth and division of cells of the T cell pathology induces cell-mediated immunity directed at the cells of the T cell pathology, or otherwise inhibits the activity of the pathological T cells. The exact mechanism that causes the alteration need not be determined, but only that an alteration in the T cell mediated pathology occurs by some mechanism following administration of the inventive molecules or compositions.

The term “T cell mediated pathology” or “T cell pathology” refers to those diseases and conditions that arise from inappropriate replication or activity of T cells. In preferred embodiments, the invention is used to treat a T cell mediated pathology that is a T cell lymphoma that results from inappropriate replication of T cells. T cell lymphomas are difficult to treat effectively with the currently available medical methods. Other types of T cell pathologies that involve inappropriate replication of T cells include chronic and acute T cell leukemias and mycosis fungoides. Other preferred embodiments include a growing number of human diseases that have been classified as autoimmune disease, where the host's own immune system attacks the host's own tissue, such as rheumatoid arthritis (RA), myasthenia gravis (MG), multiple sclerosis (MS), systemic lupus erythematosus (SLE), autoimmune thyroiditis (Hashimoto's thyroiditis), Graves' disease, inflammatory bowel disease, autoimmune uveoretinitis, polymyositis and certain types of diabetes. The present treatments for these autoimmune diseases do not cure the disease but, instead, only treat the symptoms.

The term “T cell” refers to a cell of the immune system of an organism that is involved in cell-mediated immunity in normal functioning of a organism (i.e., one that is not experiencing a T cell mediated pathology).

The term “pathology” refers to a state in an organism (e.g., a human) that is recognized as abnormal by members of the medical community. The pathologies to be treated by this invention are characterized by an abnormality in the function or replication of T cells.

The term “patient” refers to an organism in need of treatment for a pathology, or more specifically, a T cell pathology. The term refers to a living subject who has presented at a clinical setting with a particular symptom or symptoms suggesting the need for treatment with a therapeutic agent. The treatment may either be generally accepted in the medical community or it may be experimental. In preferred embodiments, the patient is a mammal, including animals such as dogs, cats, pigs, cows, sheep, goats, horses, rats, and mice. In further preferred embodiments, the patient is a human. A patient's diagnosis can alter during the course of disease progression, either spontaneously or during the course of a therapeutic regimen or treatment.

An “organism” can be single or multi-cellular. The term includes mammals, and, most preferably, humans. Preferred organisms include mice, as the ability to treat or diagnose mice is often predictive of the ability to function in other organisms such as humans. Other preferred organisms include primates, as the ability to treat or diagnose primates is often predictive of the ability to function in other organisms such as humans.

The term “chimeric protein” refers to a protein that comprises a single polypeptide chain comprising segments derived from at least two different proteins. The segments of the chimeric protein must be derived from heterologous proteins, that is, all segments of the chimeric polypeptide do not arise from the same protein. The chimeric proteins of the present invention include proteins comprising portions of the α or β chain of the TCR, but do not include the entire constant region of either the α or β chain.

The terms “protein,” “polypeptide,” “peptide” are used herein interchangeably.

The term “segment” or “portion” is used to indicate a polypeptide derived from the amino acid sequence of the proteins used as a source for the chimeric proteins having a length less than the full-length polypeptide from which it has been derived. In preferred embodiments, the segment is at least about 10, 15, 20, 21, 22, 30, 40, 70, 100, 150, 300, or 450 amino acids in length. It is understood that such segments may retain one or more characteristics of the native polypeptide. An example of such a retained characteristic is binding with an antibody specific for the native polypeptide or an epitope thereof.

The term “naturally” or “native” refers to a protein as it is isolated from nature. Thus, a naturally-occurring protein may refer to a protein as it is found in nature. A native protein may refer to a protein as it may be found or synthesized in nature. It may also apply to proteins that are produced by biological systems such as the bacculovirus virus system of the present invention or by the culture of cells of patient samples. A native protein may also refer to an isolated protein that has not been denatured. The term “native” may also refer to the manner in which polypeptide or protein is folded, either alone or in combination with other polypeptides, so that it resembles similar proteins found in nature, or how it is modified after translation (“post-translational modifications”) so that it resembles similar proteins found in nature. The only occurrence of a naturally-occurring protein may be in pathological T cells from a single patient, nevertheless, this is considered to be a naturally-occurring protein.

The terms “V_{α}” and “V_{β}” refer to the variable regions of the polypeptide chains of the TCR or nucleic acids encoding such polypeptide chains. One skilled in the art understands the meaning of these terms. The exact sequence of a variable region cannot be predicted and must be determined by isolating the sequence in question. However, multiple subfamilies of variable regions from TCR genes have been identified and characterized (See reviews by Arden et al., Immunogenetics, 1995, 42:455-500, Arden et al, Immunogenetics, 1995, 42:501-530, and Clark et al., Immunogenetics, 1995, 42:531-540). Any of these V_{α} and V_{β} regions may be used in the instant invention. The exact sequence of an alpha chain is determined by clonal rearrangements of the V regions, J regions and the Constant region of the TCR α chain locus. The exact sequence of a beta chain is determined by clonal rearrangements of the V regions, D regions, J regions and the Constant region of the TCR β chain locus. The terms “V_{α}” and “V_{β}” also refer to portions or segments of the V_{α} or V_{β} chains. A segment of a V_{α} or V_{β} chain may include at least about 30 amino acids of the V region. A segment of the V_{α} or V_{β} chain may also include all or substantially all of the V region. The term “substantially all” may refer to approximately 90% of the entire variable region, or approximately 80% of the entire variable region. The terms “V_{α}” and “V_{β}” also refer to functional derivatives of such polypeptide chains as described infra. The terms “entire V_{α} chain” refers to all of the variable region of an α chain. The term “entire V_{β} chain” refers to all of the variable region of a β chain.

The term “linker region” or “linker” refers to a segment of DNA that connects the sequence encoding the variable region, with the sequence encoding the portion of immunoglobulin constant region molecule. The linker sequence may be a synthetic sequence to allow convenient cloning, or it may be a portion of the C_{α} or C_{β} of a TCR. In either case, the linker region will not disrupt the reading frame between the portion of the variable region and the portion of the immunoglobulin constant region in a chimeric protein of the invention. The term “linker region” also includes the amino acid sequence encoded by the linker region DNA sequence. The term “synthetic linker region” or “synthetic linker” refers to a linker region that is obtained from a source other than a T-cell receptor gene or an immunoglobulin gene. In preferred embodiments, the synthetic linker is designed by a researcher and synthesized in vitro. The synthetic linker will comprise a sequence that will not break the open reading frame of the T-cell receptor and immunoglobulin gene portions of the chimeric proteins. If the linker region comprises a portion of the C_{α} or C_{β} of a TCR, it may comprise some or all of the amino acids between the start of the constant region and the first cysteine residue of the immunoglobulin fold.

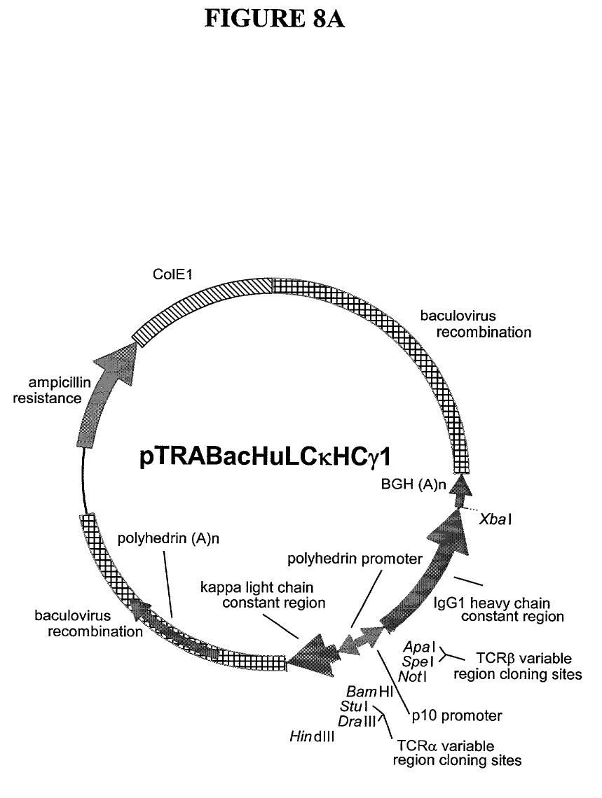

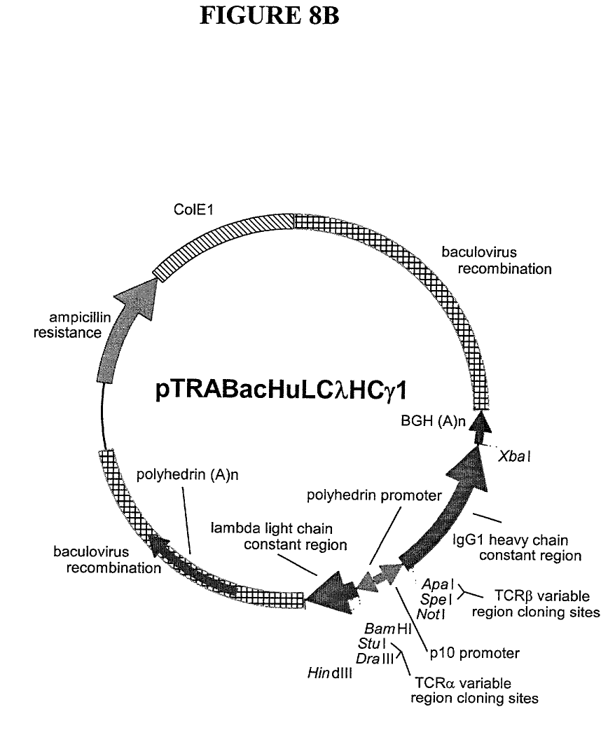

The terms “C_{α}” and “C_{β}” refer to the constant regions of the polypeptide chains of the TCR or nucleic acids encoding such polypeptide chains. One skilled in the art understands the meaning of these terms. The term “segment” or “portion,” when referring to a constant region in the present invention, does not include all of the constant region of the TCR, but may include about 3, 4, 5, 6, 7, 8, 9, 10, 14, 18, 22, 26, or 30 contiguous amino acid residues. Any integer number of codons may be present in the linker region, including any integer number from 1 to about 30, inclusive. In other preferred embodiments, up to about 80% of the amino acids of the constant region of the TCR may be present. The segment may include all or part of the first 22 amino acids of C_{α} chain up to the first cysteine of the immunoglobulin fold. A representative sequence of a human TCR alpha chain with the first cysteine of the immunoglobulin fold marked is shown in FIG. 8. The segment may include all or part of the first 30 amino acids of C_{β} chain up to the first cysteine of the immunoglobulin fold. A representative sequence of a human TCR β chain with the first cysteine of the immunoglobulin fold marked is shown in FIG. 8. The terms “C_{α}” and “C_{β}” also refer to functional derivatives of such polypeptide chains as described infra.

The term “TCR” or “T cell receptor” refers to a polypeptide found on the surface of T cells that comprises two polypeptide chains, and alpha chain and a beta chain. The term “TCR” or “T cell receptor” may also refer to nucleic acids encoding such polypeptide chains. Due to the normal development of the immune system, TCRs display considerable sequence diversity due to the operation of DNA rearrangements such as described in Bell et al. (Bell et al., 1995, T Cell Receptors, Oxford University Press, Oxford). The exact sequence of a given TCR cannot be predicted and must be determined by sequencing either the encoding nucleic acid or the protein of the TCR in question. Any of the potential sequences of TCRs may be used in the instant invention.

The term “immunoglobulin constant region” refers to all or part of that portion of immunoglobulin molecules that are not encoded by the variable regions of immunoglobulins. The term “immunoglobulin constant region” may also refer to the DNA sequence encoding the immunoglobulin constant region. The immunoglobulin constant region includes the segments C_{L}, C_{H1}, C_{H2}, C_{H3}, and the Hinge region. Immunoglobulin types include IgG₁, IgG₂, IgG₃, IgG₄, IgA, IgA₁, IgA₂, IgM, IgD, IgE heavy chains, and κ or λ light chains or segments thereof. In preferred embodiments, the heavy and light chain constant regions are derived from 9F12 cells. Any immunoglobulin constant region segments from any source may be used in the instant invention, provided that the segment allows for the affinity purification of the chimeric molecule, for example, via binding to Protein G, Protein A, Protein L, or an appropriate antibody. Functional derivatives of the immunoglobulin constant region segments, as described infra, may also be used.

The term “immunoglobulin fold” or “immunoglobulin domain” refers to a structural element of the immunoglobulin super family. The immunoglobulin domain is a conserved, repeating structural domain of approximately 110 amino acids each. Within each domain, in general, there are seven antiparallel β strands, arranged in two sheets of four and three strands respectively, and an intrachain disulfide bond that forms a loop of about 60 amino acids. The two sheets generally face each other so that the hydrophobic amino acids face inwards, and the hydrophilic amino acids face outwards.

Immunoglobulin domains are found in many protein molecules, including antibodies, the T cell antigen receptor, cytokine receptors (e.g. the platelet-derived growth factor receptor with 5 Ig domains), cell adhesion molecules (e.g. ICAM-1/CD54), and many others. Two immunoglobulin domains are found in each chain of a TCR; one in the variable region and one in the constant region. Two immunoglobulin domains are found in antibody light chains and four are found in IgG heavy chains.

The terms “kappa constant region,” “lambda constant region,” “κ constant region,” and “λ constant region” refer to the constant regions of kappa (κ) and lambda (λ) light chains that remain constant during the development of the immune system. The terms may refer to either the DNA sequences or the amino acid sequences of the proteins. In some embodiments, the immunoglobulin light chain may be comprised in a chimeric protein that contains amino acids from one or more other proteins.

The terms “IgG₁, IgG₂, IgG₃, IgG₄, IgA, IgA₁, IgA₂, IgM, IgD, IgE” refer to classes and subclasses of human immunoglobulins. The terms may refer to either the DNA sequences or the amino acid sequences of the proteins. The class and subclass of an immunoglobulin molecule is determined by its heavy chain. For example, IgG and IgD are different classes of immunoglobulins; IgG₁ and IgG₂ are different subclasses of immunoglobulin molecules. The term “IgA” may refer to any subclass of IgA molecules. In preferred embodiments, it refers to an IgA₁ molecule. In other preferred embodiments, it refers to an IgA₂ molecule. In some embodiments, the immunoglobulin heavy chain used may be a chimeric protein that contains amino acids from a second protein.

The term “IgG_{γ1}” refers to the heavy chain associated with the IgG₁ class of immunoglobulins. IgG₁ represents approximately 66% of human IG immunoglobulins (Roitt et al., Immunology, Mosby, St. Louis, 1993, pg. 4.2).

The term “administering” relates to a method of contacting a compound with or into cells or tissues of an organism. The T cell mediated pathology can be prevented or treated when the cells or tissues of the organism exist within the organism or outside of the organism. Cells existing outside the organism can be maintained or grown in cell culture dishes. For cells harbored within the organism, many techniques exist in the art to administer compounds, including (but not limited to) oral, parenteral, dermal, injection, and aerosol applications. The effect of administering a compound on organism function can then be monitored. The organism is preferably a mouse, rat, rabbit, guinea pig, or goat, more preferably a monkey or ape, and most preferably a human.

The term “composition” refers to a mixture that contains the protein of interest. In preferred embodiments, the composition may contain additional components, such as adjuvants, stabilizers, excipients, and the like.

The term “associated with” in reference to the relation of a variable region to a T cell clone refers to the variable region that is found on the immunoglobulins produced by a particular T cell clone.

The term “T cell clone” refers to the clonal descendants of a single T cell. Clonal descendants of T cells express the same idiotype in the produced TCR as the parental cell. One skilled in the art realizes that clonal descendants of a T cell may have undergone somatic mutation within the TCR gene but still remain part of the T cell clone.

The term “isolating” refers to removing a naturally occurring nucleic acid sequence from its normal cellular environment. Thus, the sequence may be in a cell-free solution or placed in a different cellular environment. The term does not imply that the sequence is the only nucleotide chain present, but that it is essentially free (about 90-95% pure at least) of non-nucleotide material naturally associated with it, and thus is distinguished from isolated chromosomes. Also, by the use of the term “isolating” in reference to nucleic acid is meant that the specific DNA or RNA sequence is increased to a significantly higher fraction (2- to 5-fold) of the total DNA or RNA present in the solution of interest than in the cells from which the sequence was taken. This could be caused by a person by preferential reduction in the amount of other DNA or RNA present, or by a preferential increase in the amount of the specific DNA or RNA sequence, or by a combination of the two. However, it should be noted that enriched does not imply that there are no other DNA or RNA sequences present, just that the relative amount of the sequence of interest has been significantly increased. The term “significant” is used to indicate that the level of increase is useful to the person making such an increase, and generally means an increase relative to other nucleic acids of about at least 2-fold, more preferably at least 5- to 10-fold or even more. The term also does not imply that there is no DNA or RNA from other sources. The DNA from other sources may, for example, comprise DNA from a yeast or bacterial genome, or a cloning vector such as pUC19. This term distinguishes from naturally occurring events, such as viral infection, or tumor-type growths, in which the level of one mRNA may be naturally increased relative to other species of mRNA. That is, the term is meant to cover only those situations in which a person has intervened to elevate the proportion of the desired nucleic acid.

Isolated DNA sequences are relatively more pure than in the natural environment (compared to the natural level this level should be at least 2- to 5-fold greater, e.g., in terms of mg/mL). Individual sequences obtained from PCR may be purified to electrophoretic homogeneity. The DNA molecules obtained from this PCR reaction could be obtained from total DNA or from total RNA. These DNA sequences are not naturally occurring, but rather are preferably obtained via manipulation of a partially purified naturally occurring substance (e.g., messenger RNA (mRNA)). For example, the construction of a cDNA library from mRNA involves the creation of a synthetic substance (cDNA) and pure individual cDNA clones can be isolated from the synthetic library by clonal selection from the cells carrying the cDNA library. The process that includes the construction of a cDNA library from mRNA and isolation of distinct cDNA clones yields an approximately 10⁶-fold purification of the native message. Thus, purification of at least one order of magnitude, preferably two or three orders, and more preferably four or five orders of magnitude is expressly contemplated.

The term “gene encoding” refers to a sequence of nucleic acids that codes for a protein or polypeptide of interest. The nucleic acid sequence may be either a molecule of DNA or RNA. In preferred embodiments, the molecule is a DNA molecule. In other preferred embodiments, the molecule is a RNA molecule. When present as a RNA molecule, it will comprise sequences that direct the ribosomes of the host cell to start translation (e.g., a start codon, ATG) and direct the ribosomes to end translation (e.g., a stop codon). Between the start codon and stop codon is an open reading frame (ORF). One skilled in the art is very familiar with the meaning of these terms.

The term “insect cell lines” refers to cell lines derived from insects and susceptible to infection by the bacculovirus. One skilled in the art is familiar with such cell lines and the techniques needed to utilize them. Representative examples of insect cell lines include Spodoptera frugiperda (sf9) and Trichoplusia ni (Hi-5) cell lines.

The terms “Trichoplusia ni (High-5) cells and “Spodoptera frugiperda (sf9) cells” refers to insect cell lines used in combination with baculovirus expression vectors. One skilled in the art is familiar with these cell lines and how to obtain them.

The term “adjuvant” refers to a substance which is provided with the antigen or immunogen of choice, e.g., the protein or polypeptide to which an immune response is desired, to enhance the immune response when one attempts to raise an immune response in an animal against the antigen or immunogen of choice. One skilled in the art is familiar with appropriate adjuvants to select and use. Adjuvants approved for human use include aluminum salts and MF59 (Singh and O'Hagan, Nature Biotech 17:1075-81, 1999). Other adjuvants are being developed (Id.) and may be used in conjunction with the present invention.

The term “keyhole-limpet hemocyanin” refers to a protein which is isolated from keyhole limpets that is commonly used as a carrier protein in the immunization process. One skilled in the art is familiar with the meaning of the term keyhole limpet hemocyanin.

The term “cytokine” refers to a family of growth factors, usually soluble (glyco)proteins, secreted primarily from leukocytes. Cytokines stimulate both the humoral and cellular immune responses, as well as the activation of phagocytic cells. Cytokines are synthesized, stored and transported by various cell types not only inside of the immune system (lymphokines, interleukins, monokines, tumor necrosis factors, interferons) but also by other cells that are studied in hematology (producing colony-stimulating factors), oncology (producing transforming growth factors), and cell biology (producing peptide growth factors, heat shock and other stress proteins).

Cytokines secreted from lymphocytes are termed lymphokines, while those secreted by monocytes or macrophages are referred to as monokines. Many of the lymphokines are also referred to as interleukins (ILs), since they are not only secreted by leukocytes but they are also able to affect the cellular responses of leukocytes. Specifically, interleukins function as growth factors targeted to cells of hematopoietic origin.

The term “growth factor” refers to a protein that binds receptors on the surface of a cell and subsequently activates cellular proliferation and/or differentiation. Many growth factors are quite versatile and can act to stimulate cellular division in a wide variety of cell types, while others are specific to a particular cell-type.

The term “chemokine” refers to a group of small proinflammatory cytokines that function as chemoattractants and activators for leukocytes and represent a superfamily of over thirty chemotactic cytokines. They orchestrate the activation and migration of immune system cells from the blood or bone marrow to the site of infection and damaged tissue. Chemokines also play an essential role in the growth and proliferation of primitive stem cells found in bone marrow, which in turn grow into mature immune cells. They are involved in a wide range of acute and inflammatory diseases and primarily exert their action by binding to receptors of the seven-transmembrane-helix class.

Chemokines frequently range from 8 to 11 kDa in molecular weight, are active over a concentration range of 1 to 100 ng/ml, and are produced by a wide variety of cell types. The production of chemokines is induced by exogenous irritants and endogenous mediators such as IL-1, TNF-alpha, and PDGF. The chemokines bind to specific cell surface receptors and can be considered second-order cytokines that appear to be less pleiotropic than first-order proinflammatory cytokines because they are not potent inducers of other cytokines and exhibit more specialized functions in inflammation and repair.

The term “granulocyte-macrophage colony-stimulating factor “or “GM-CSF” refers to a small (less than 20 kDa) secreted protein. It binds to specific cell surface receptors and functions as species-specific stimulator of bone marrow cells. It stimulates the growth and differentiation of several hematopoietic cell lineages including dendritic cells, granulocytes, macrophages, eosinophils, and erythrocytes. In particular, this cytokine also plays a role in shaping cellular immunity by augmenting T-cell proliferation (Santoli et al, J.Immunol, 1988, 141(2):519-26), increasing expression of adhesion molecules on granulocytes and monocytes (Young et al., J.Immunol, 1990, 145(2):607-15; Grabstein et al., Science, 1986, 232(4749):506-08), and by augmenting antigen presentation (Morrissey et al., J.Immunol, 1987, 139(4):1113-9; Heufler et al., J.Exp.Med., 1988, 167(2):700-05; Smith et al., J.Immunol, 1990, 144(5):1777-82).

The term “monocyte chemotactic protein-3” or “MCP-3” refers a chemokine primarily produced by monocytes. MCP-3 has a wide spectrum of chemotactic activity and attracts monocytes, dendritic cells, lymphocytes, natural killer cells, eosinophils, basophils, and neutrophils. The cDNA was cloned in 1993 by Minty et al., Eur Cytokine Netw 4(2):99-110, 1993, and Opdenakker et al., Biochem Biophys Res Commun., 191(2):535-42, 1993. Its properties have been recently reviewed by Proost et al., J Leukoc Biol. 59(1):67-74, 1996.

The term “expression vector” refers to a recombinant DNA construct that is designed to express a selected gene of interest, usually a protein, when properly inserted into the expression vector. One skilled in the art understands the term. Expression vectors commonly include a promotor at the 5′ end of the site where the gene of interest is inserted and a terminator region at 3′ end of the site. Frequently the gene of interest is inserted into the appropriate site by means of selected restriction enzyme cleavage sites. The term “expression vector” also refers to a DNA construct such as described above into which the gene of interest encoding the product of interest has already been inserted.

The term “baculovirus expression vector” refers to a DNA construct that is designed to express a selected gene when used in the baculovirus system. Any of the potential baculoviruses or expression vectors designed to function in the baculovirus system may be used in the instant invention. In a similar fashion, the term “expression vector” is a genus that encompasses the particular embodiment of baculovirus expression vectors, but “expression vectors” may function in cells and cell lines aside from, or in addition to, insect cell lines.

The term “allow the expression of” refers to placing an expression vector into an environment in which the gene of interest will be expressed. This commonly means inserting the expression vector into an appropriate cell type where the promotor and other regions necessary for gene expression will be recognized by the host cell's components and will cause the expression of the gene of interest. The expression normally consists of two steps: transcription and translation. Expression can also be conducted in vitro using components derived from cells. One skilled in the art is familiar with these techniques, and such techniques are set forth in Sambrook et al. (Sambrook, Fritsch, & Maniatis, “Molecular Cloning: A Laboratory Manual”, 2nd ed., Cold Spring Harbor Laboratory, 1989). In the preferred embodiment, the expressed product is a protein or polypeptide. In other preferred embodiments, the expressed product is V_{β}/IgG_{γ1}, V_{β}/C_{κ}, V_{β}/C_{κ}, V_{α}/C_{λ}, V_{α}/C_{λ}, or V_{α}/IgG_{γ1}.

The term “secretory signal sequence” refers to a peptide sequence. When this sequence is translated in frame as a peptide attached to the amino-terminal end of a polypeptide of choice, the secretory signal sequence will cause the secretion of the polypeptide of choice by interacting with the machinery of the host cell. As part of the secretory process, this secretory signal sequence will be cleaved off, leaving only the polypeptide of interest after it has been exported. In preferred embodiments, the honey bee melittin secretory signal sequence is employed. In other preferred embodiments, the human placental alkaline phosphatase secretory signal sequence is employed. In further preferred embodiments, the endogenous secretory sequences associated with the immunoglobulin genes derived from a given patient are used. The present invention, however, is not limited by these secretory signal sequences and others well known to those skilled in the art may be substituted in place of, and in addition to, these. The term “secretory signal sequence” also refers to a nucleic acid sequence encoding the secretory peptide.

The term “ELISA” refers to “Enzyme-Linked ImmunoSorbent Assay” in which the presence or concentration of a protein is determined by its binding to the plastic well of an ELISA plate followed by its subsequent detection by antibodies specific for the protein to be quantified or detected.

The term “promoter controls” refers to an arrangement of DNA in an expression vector in which a promoter is placed 5′ to a gene of interest and causes the transcription of the DNA sequence into an mRNA molecule. This mRNA molecule is then translated by the host cell's machinery. One skilled in the art is very familiar with the meaning of this term.

The terms “protein A,” “protein G,” and “protein L” refer to specific bacterial proteins which are capable of specifically binding immunoglobulin molecules without interacting with an antigen binding site. Protein A is a polypeptide isolated from Staphylococcus aureus that binds the Fc region of immunoglobulin molecules. Protein G is a bacterial cell wall protein with affinity for immunoglobulin G (IgG), which has been isolated from a human group G streptococcal strain (G148). Protein L is an immunoglobulin light chain-binding protein expressed by some strains of the anaerobic bacterial species Peptostreptococcus magnus.

The term “isolating” as refers to a protein or polypeptide, refers to removing a naturally occurring polypeptide or protein from its normal cellular environment or refers to removing a polypeptide or protein synthesized in an expression system (such as the baculovirus system described herein) from the other components of the expression system. Thus, the sequence may be in a cell-free solution or placed in a different cellular environment. The term does not imply that the sequence is the only amino acid chain present, but that it is essentially free (about 90-95% pure at least) of non-amino acid-based material naturally associated with it.

By the use of the term “enriched” in reference to a polypeptide is meant that the specific amino acid sequence constitutes a significantly higher fraction (2- to 5-fold) of the total amino acid sequences present in the cells or solution of interest than in normal or diseased cells or in the cells from which the sequence was taken. This could be caused by a person by preferential reduction in the amount of other amino acid sequences present, or by a preferential increase in the amount of the specific amino acid sequence of interest, or by a combination of the two. However, it should be noted that enriched does not imply that there are no other amino acid sequences present, just that the relative amount of the sequence of interest has been significantly increased. The term significant here is used to indicate that the level of increase is useful to the person making such an increase, and generally means an increase relative to other amino acid sequences of about at least 2-fold, more preferably at least 5- to 10-fold or even more. The term also does not imply that there is no amino acid sequence from other sources. The other source of amino acid sequences may, for example, comprise amino acid sequence encoded by a yeast or bacterial genome, or a cloning vector such as pUC19. In preferred embodiments, the amino acid sequence is a chimeric protein as described above. The term is meant to cover only those situations in which man has intervened to increase the proportion of the desired amino acid sequence.

It is also advantageous for some purposes that an amino acid sequence be in purified form. The term “purified” in reference to a polypeptide does not require absolute purity (such as a homogeneous preparation); instead, it represents an indication that the sequence is relatively purer than in the natural environment. Compared to the natural level this level should be at least 2-to 5-fold greater (e.g., in terms of mg/mL). Purification of at least one order of magnitude, preferably two or three orders, and more preferably four or five orders of magnitude is expressly contemplated. The substance is preferably free of contamination at a functionally significant level, for example 90%, 95%, or 99% pure.

The term “operatively linked” refers to an arrangement of DNA in which a controlling region, such as a promoter or enhancer, is attached to a connected DNA gene of interest so as to bring about its transcription, and hence allowing its translation. The term “operatively linked” may also refer to a DNA sequence encoding a processing signal, such as a secretory signal sequence, connected to a gene encoding a polypeptide to form a single open reading frame. Following transcription and translation, the secretory signal sequence has the potential to bring about the export of the translated polypeptide. One skilled in the art is familiar with the meaning of this term.

Functional Derivatives of Useful Chimeric Proteins

Also provided herein are functional derivatives of a polypeptide or nucleic acid of the invention. By “functional derivative” is meant a “chemical derivative,” “fragment,” or “variant,” of the polypeptide or nucleic acid of the invention; these terms are defined below. A functional derivative retains at least a portion of the function of the protein, for example reactivity with an antibody specific for the protein, enzymatic activity or binding activity mediated through noncatalytic domains, which permits the utility of the functional derivative in accordance with the present invention. It is well known in the art that due to the degeneracy of the genetic code numerous different nucleic acid sequences can code for the same amino acid sequence. Equally, it is also well known in the art that conservative changes in amino acid can be made to arrive at a protein or polypeptide that retains the functionality of the original. In both cases, all permutations are intended to be covered by this disclosure.

Included within the scope of this invention are the functional equivalents of the herein-described isolated nucleic acid molecules. The degeneracy of the genetic code permits substitution of certain codons by other codons that specify the same amino acid and hence would give rise to the same protein. The nucleic acid sequence can vary substantially since, with the exception of methionine and tryptophan, the known amino acids can be coded for by more than one codon. Thus, portions or all of the genes of the invention could be synthesized to give a nucleic acid sequence significantly different from a sequence that is found in nature. The encoded amino acid sequence thereof would, however, be preserved.

A “chemical derivative” of the complex contains additional chemical moieties not normally a part of the protein. Covalent modifications of the protein or peptides are included within the scope of this invention. Such modifications may be introduced into the molecule by reacting targeted amino acid residues of the peptide with an organic derivatizing agent that is capable of reacting with selected side chains or terminal residues, as described below.

Cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylprocarbonate at pH 5.5-7.0 because this agent is relatively specific for the histidyl side chain. Para-bromophenacyl bromide also is useful; the reaction is preferably performed in 0.1 M sodium cacodylate at pH 6.0.

Lysinyl and amino terminal residues are reacted with succinic or other carboxylic acid anhydrides. Derivatization with these agents has the effect or reversing the charge of the lysinyl residues. Other suitable reagents for derivatizing primary amine containing residues include imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKₐ of the guanidine functional group. Furthermore, these reagents may react with the groups of lysine as well as the arginine α-amino group.

Tyrosyl residues are well-known targets of modification for introduction of spectral labels by reaction with aromatic diazonium compounds or tetranitromethane. Most commonly, N-acetylimidizol and tetranitromethane are used to form O-acetyl tyrosyl species and 3-nitro derivatives, respectively.

Carboxyl side groups (aspartyl or glutamyl) are selectively modified by reaction with carbodiimide (R′—N—C—N—R′) such as 1-cyclohexyl-3-(2-morpholinyl(4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Glutaminyl and asparaginyl residues are frequently deamidated to the corresponding glutamyl and aspartyl residues. Alternatively, these residues are deamidated under mildly acidic conditions. Either form of these residues falls within the scope of this invention.

Derivatization with bifunctional agents is useful, for example, for cross-linking the component peptides of the protein to each other or to other proteins in a complex to a water-insoluble support matrix or to other macromolecular carriers. Commonly used cross-linking agents include, for example, 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[p-azidophenyl] dithiolpropioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Other modifications include hydroxylation of proline and lysine, phosphorylation of hydroxyl groups of seryl or threonyl residues, methylation of the α-amino groups of lysine, arginine, and histidine side chains (Creighton, T. E., Proteins: Structure and Molecular Properties, W.H. Freeman & Co., San Francisco, pp. 79-86 (1983)), acetylation of the N-terminal amine, and, in some instances, amidation of the C-terminal carboxyl groups.

Such derivatized moieties may improve the stability, solubility, absorption, biological half life, and the like. The moieties may alternatively eliminate or attenuate any undesirable side effect of the protein complex and the like. Moieties capable of mediating such effects are disclosed, for example, in Remington's Pharmaceutical Sciences, 18th ed., Mack Publishing Co., Easton, Pa. (1990).

A functional derivative of a protein with deleted, inserted and/or substituted amino acid residues may be prepared using standard techniques well-known to those of ordinary skill in the art. For example, the modified components of the functional derivatives may be produced using site-directed mutagenesis techniques (as exemplified by Adelman et al., 1983, DNA 2:183) wherein nucleotides in the DNA coding the sequence are modified such that a modified coding sequence is modified, and thereafter expressing this recombinant DNA in a prokaryotic or eukaryotic host cell, using techniques such as those described above. Alternatively, proteins with amino acid deletions, insertions and/or substitutions may be conveniently prepared by direct chemical synthesis, using methods well-known in the art. The functional derivatives of the proteins typically exhibit the same qualitative biological activity as the native proteins.

The term “fragment” is used to indicate a polypeptide derived from the amino acid sequence of a protein having a length less than the full-length polypeptide from which it has been derived. Such a fragment may, for example, be produced by proteolytic cleavage of the full-length protein. Preferably, the fragment is obtained recombinantly by appropriately modifying the DNA sequence encoding the proteins to delete one or more amino acids at one or more sites of the C-terminus, N-terminus, and/or within the native sequence. It is understood that such fragments may retain one or more characterizing portions, functions, or characteristics of the native protein or polypeptide. Examples of such retained characteristics include: catalytic activity; substrate specificity; interaction with other molecules in the intact cell; regulatory functions; or binding with an antibody specific for the native complex, or an epitope thereof.

Another functional derivative intended to be within the scope of the present invention is a “variant” polypeptide which either lacks one or more amino acids or contains additional or substituted amino acids relative to the native polypeptide. The variant may be derived from a naturally occurring complex component by appropriately modifying the protein DNA coding sequence to add, remove, and/or to modify codons for one or more amino acids at one or more sites of the C-terminus, N-terminus, and/or within the native sequence. It is understood that such variants having added, substituted and/or additional amino acids retain one or more characterizing portions of the native protein, as described supra.

Other aspects of the invention relate to uses for the instant chimeric proteins. Preferred uses include pharmaceutical and veterinary applications, wherein an effective amount of chimeric protein according to the invention (preferably in a composition according hereto) is administered to a patient. In this way, the chimeric protein contacts cells of the patient, which contacting thereafter elicits the desired biological response. Methods for using the instant chimeric proteins include methods of eliciting an immune response in an organism, methods of raising antibodies (B cell immune response) in an organism, methods of inducing a T cell immune response by an organism, and methods for treating T cell pathologies. The invention also includes methods for treatment of subjects in order to increase the immune response capable of altering a T cell pathology by administering a chimeric protein of the invention.

Typically, such methods are accomplished by delivering to the organism an effective amount of a chimeric protein according to the invention. “Effective amount” refers to an amount that results in the desired biological response being elicited. What constitutes such an amount will vary, and depends on a variety of factors, including the particular chimeric protein, the desired biological response to be elicited, the formulation of the chimeric protein, the age, weight, gender, and health of the organism to be treated, the dosage regimen, the condition or disease to be treated or prevented, etc. Organisms to which the instant chimeric proteins and compositions may be administered include mammals, preferably a mammal selected from the group consisting of a bovine, canine, equine, feline, ovine, porcine, and primate animal. Particularly preferred organisms are humans.

The compounds described herein can be administered to a human patient per se, or in pharmaceutical compositions where it is mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., latest edition.

Routes of Administration

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections. One of skill in the art will understand the various modifications that would be made to adapt the composition to a particular route of administration.

Alternately, one may administer the compound in a local rather than systemic manner, for example, via injection of the compound directly into a solid tumor, often in a depot or sustained release formulation.

Composition/Formulation

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Suitable carriers include excipients such as, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxyrnethyl cellulose, sorbitol, or dextran. Optionally, the suspension may also contain suitable stabilizers or agents that increase the solubility of the compounds to allow for the preparation of highly concentrated solutions.

Alternatively, the active ingredient may be in powder form for constitution with a suitable vehicle, e.g., sterile pyrogen-free water, before use.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:D5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

Effective Dosage

Pharmaceutical compositions suitable for use in the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

Toxicity and therapeutic efficacy of the compounds described herein can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds that exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics”, Ch. 1 p. 1).

Dosage amount and interval may be adjusted individually to provide plasma levels of the active moiety that are sufficient to maintain the required effect, or minimal effective concentration (MEC). The MEC will vary for each compound. Dosages necessary to achieve the MEC will depend on individual characteristics and route of administration. However, HPLC assays or bioassays can be used to determine plasma concentrations.

Dosage intervals can also be determined using MEC value. Compounds should be administered using a regimen that maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

Packaging

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the polynucleotide for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition. Suitable conditions indicated on the label may include treatment of a tumor, treatment of rheumatoid arthritis, treatment of diabetes, and the like.

### EXAMPLES

In the following description, reference will be made to various methodologies known to those skilled in the art of immunology, cell biology, and molecular biology. Publications and other materials setting forth such known methodologies to which reference is made are incorporated herein by reference in their entireties as though set forth in full.

### Example 1

### Tissue Processing for T cell Lymphoma Idiotype (Id) Identification and Cloning

Tumor samples from a peripheral lymph node are biopsied as clinically indicated under sterile conditions and used to generate patient idiotype-specific recombinant V_{α} and V_{β}-Ig chimeric proteins. Remaining lymph node biopsy material is stored in liquid nitrogen in tissue cell bank for future use.

Cell Isolation:

Single cell suspensions of patient lymph node biopsies are obtained by forcing the biopsied lymphoma tissue through a disposable 0.38 mm steel mesh screen, while submerged in sterile PBS. The dispersed cells are washed twice in PBS, then resuspended and counted. A 10% fraction of the cells are processed for total RNA extraction and the remaining cells are archived in liquid nitrogen following resuspension in RPMI 1640 tissue culture media containing 30% fetal bovine serum and 10% DMSO. All processing of clinical samples is performed in a biological safety cabinet.

Total RNA Preparation:

Total RNA from homogenized lymph node cells is isolated using RNeasy Kit (Qiagen) as per manufacturer's instruction. Total RNA is quantitated by spectrophotometry.

cDNA Synthesis and PCR Amplification of Genes Encoding V_{α} and V_{β} Chains of a TCR from T Lymphoma Cells:

Both V_{α} and V_{β} chains of a T lymphoma cell receptor are identified using the 5′ RACE system (Gibco BRL) exactly as described by the manufacturer with the following specifics: The C_{α} specific primer CADS3, or C_{β} specific primer CBDS3, (see Table 1) are used to prime first strand DNA using approximately 5.0 μg total RNA as template for first strand cDNA synthesis. Poly dC-tailing was performed as per manufacturer's recommendation. V_{α} and V_{β} identification was then performed using a 5′ primer provided by manufacturer and a second C_{α} specific CADS2 or C_{β} specific CBDS2 antisense primer, each internal to the respective primer used for cDNA synthesis (see Table 1).

TABLE 1
PRIMER SEQUENCES

| PRIMER NAME | PRIMER SEQUENCE (5′ 3′) | |
|---|---|---|
| 1. CADS2 | 5′CATCAGAATCCTTACTTTGTGACAC3′ | SEQ ID NO:10 |
| 2. CADS3 | 5′CCATAGACCTCATGTCTAGCACAG3′ | SEQ ID NO:11 |
| 3. CBDS2 | 5′CTGTGCACCTCCTTCCCATTCAC3′ | SEQ ID NO:12 |
| 4. CBDS3 | 5′GGCAGTATCTGGAGTCATTGAGG3′ | SEQ ID NO:13 |
| 5. CB/IgG₁ | 5′GCGACCTCGGGTGGGccCACC/GTTG/TTTCAGG3′ | SEQ ID NO:14 |
| 6. CA/IgK | 5′CAGCTGGTACACcaCttGGTgAGGGTTCTGGAT3′ | SEQ ID NO:15 |

(CB/IgG1: letters in small letters denote changes to create Apa I cloning site to form IgG₁ chimeric protein. Bases designated C/G and G/T designate either base for use at those two positions due to differences between C_{β}1 & 2.)
(CA/IgK: letters in small letters denote bp changes to create Dra III cloning site to form chimeric protein with a Kappa light chain.)

Cloning and Sequencing of PCR Products:

PCR products from reactions determined to contain the tumor specific sequences for V_{α} and V_{β} chains are cloned directly into plasmid pCR2. 1-TOPO as per manufacturer's recommendations, and introduced into Top10 competent E. coli cells (Invitrogen). Twenty-four miniprep DNA plasmids are prepared from carbenicillin resistant bacterial colonies using the QIAPrep Spin Miniprep Kit (Qiagen), and quantitated by spectrophotometry. Two hundred ng of each plasmid was sequenced using the Cy5/Cy5.5 Dye Primer Cycle Sequencing Kit (Visible Genetics). Following the completion of the sequencing reactions, samples were electrophoresed on the OpenGene Automated DNA Sequencing System and the data was processed with GeneObjects software package (Visible Genetics). Additional analysis including sequence alignments were performed using the SEQUENCHER Version 4.1.2 DNA analysis software (GENE Codes Corp.). A tumor specific sequence for V_{α} or V_{β} would be considered if it is present in 75% of the samples, for example, if 18 or greater of the 24 form a consensus group. Two independent biopsy samples are compared when available.

### Example 2

### Construction of Baculovirus Expression Vectors pTRABacV₆₀ HC_{γ1} and pTRABacV_{β}HC_{γ1} Containing Constant Regions of Immunoglobulin Heavy and Light Chains

Cloning of Secretory Signal Sequences into p2Bac:

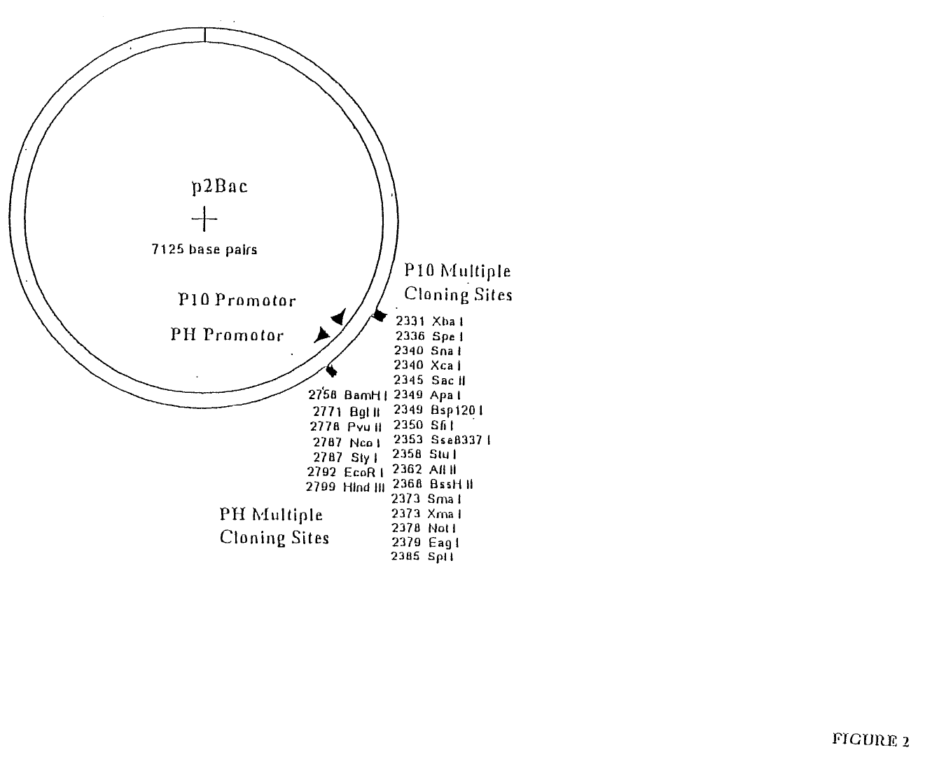

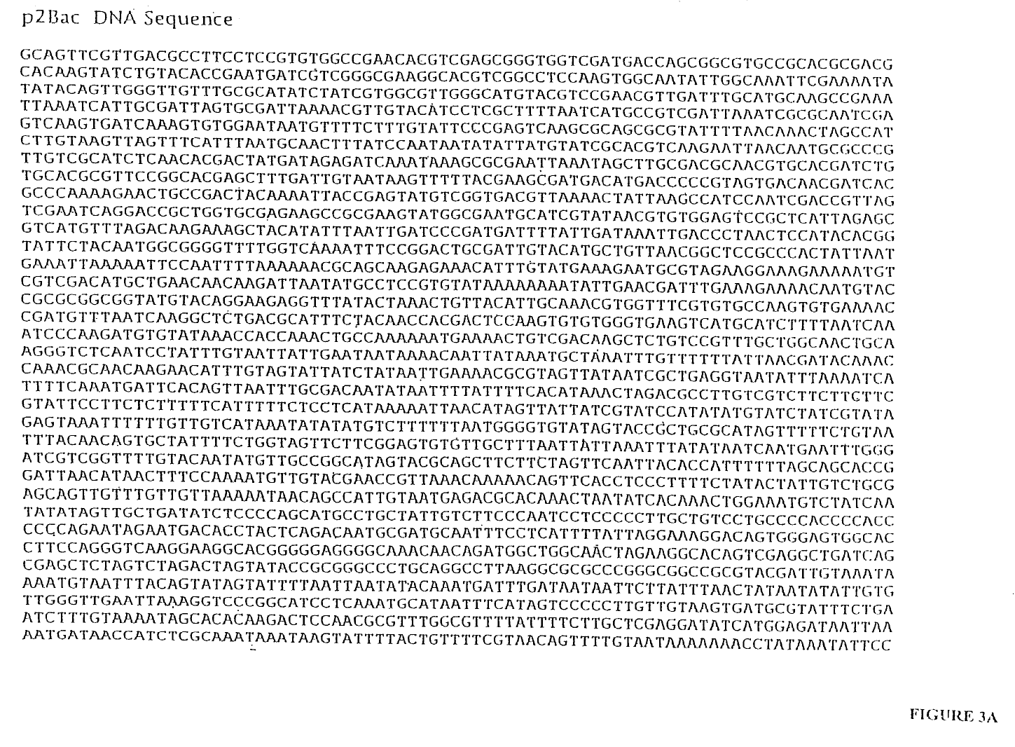

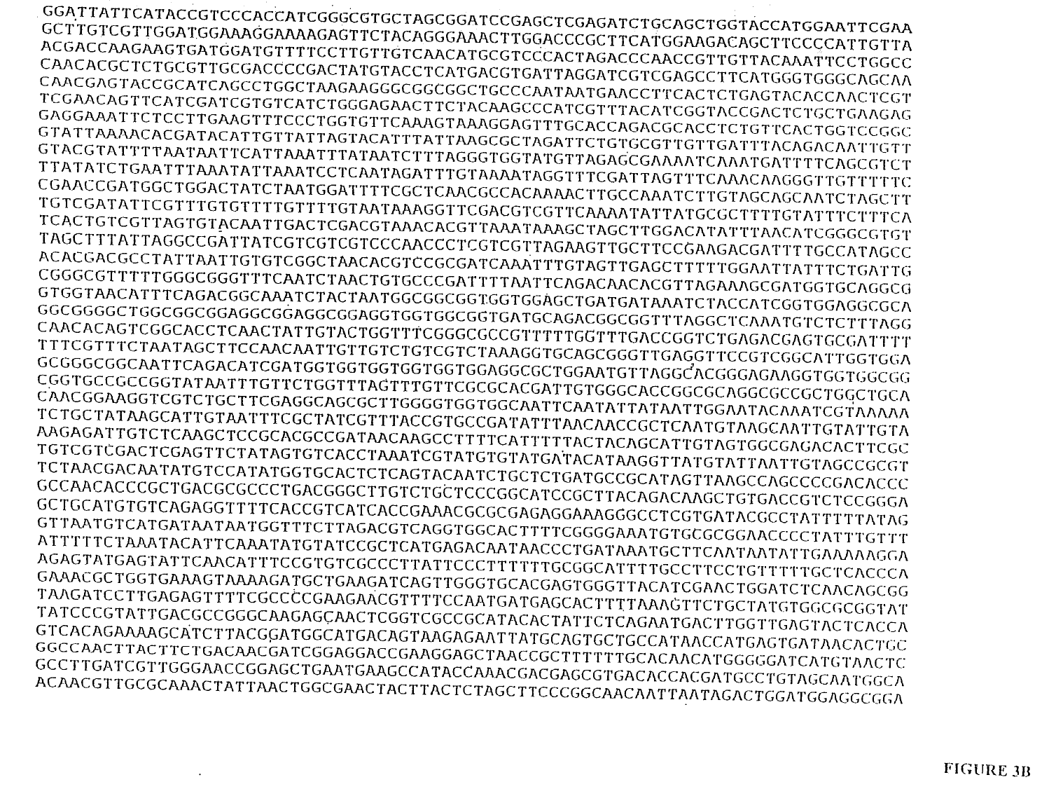

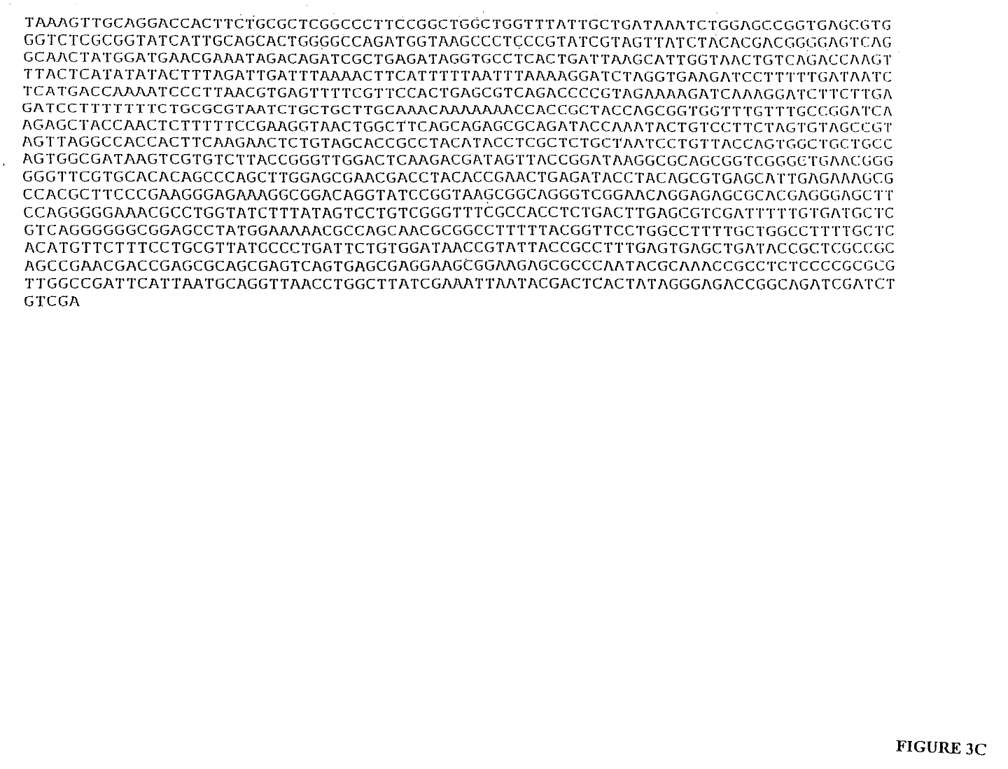

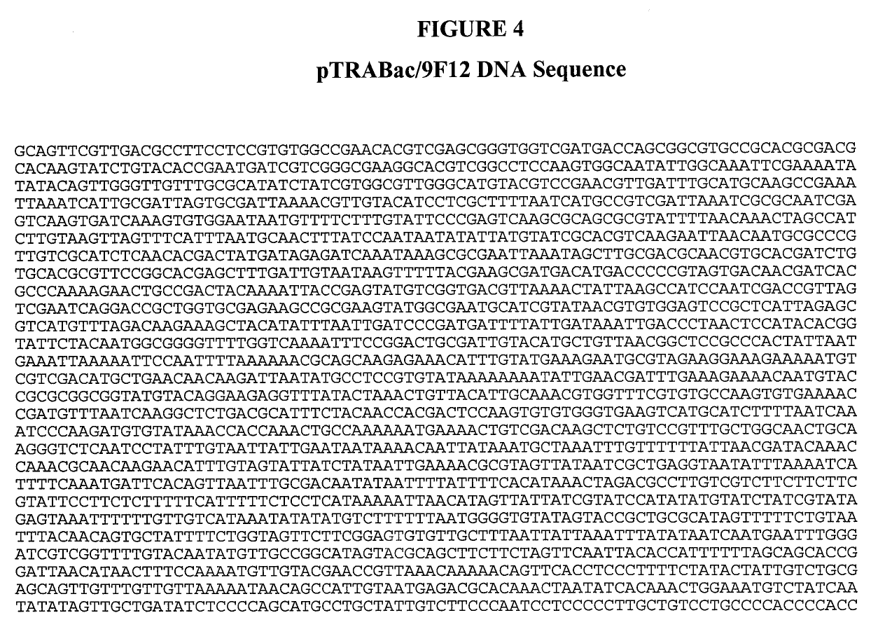

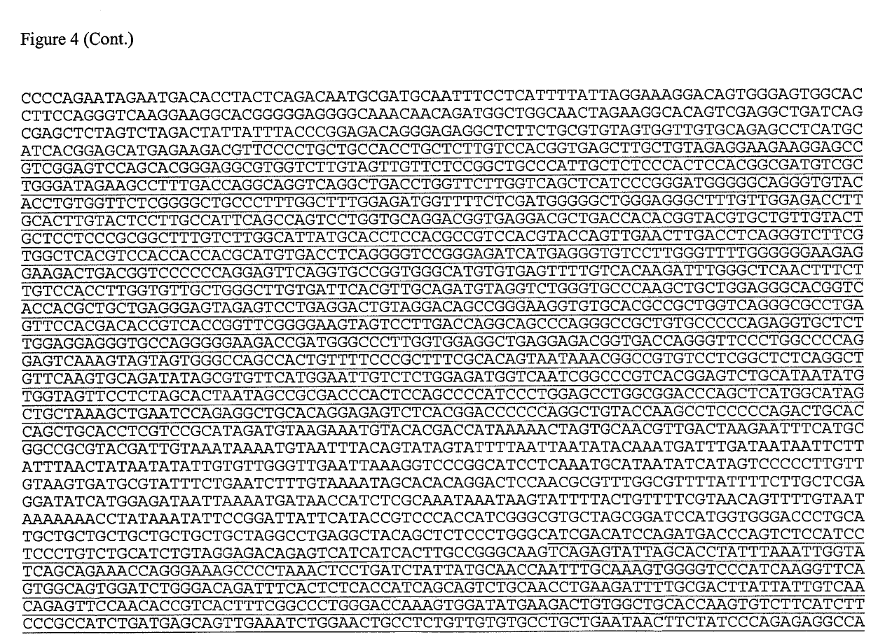

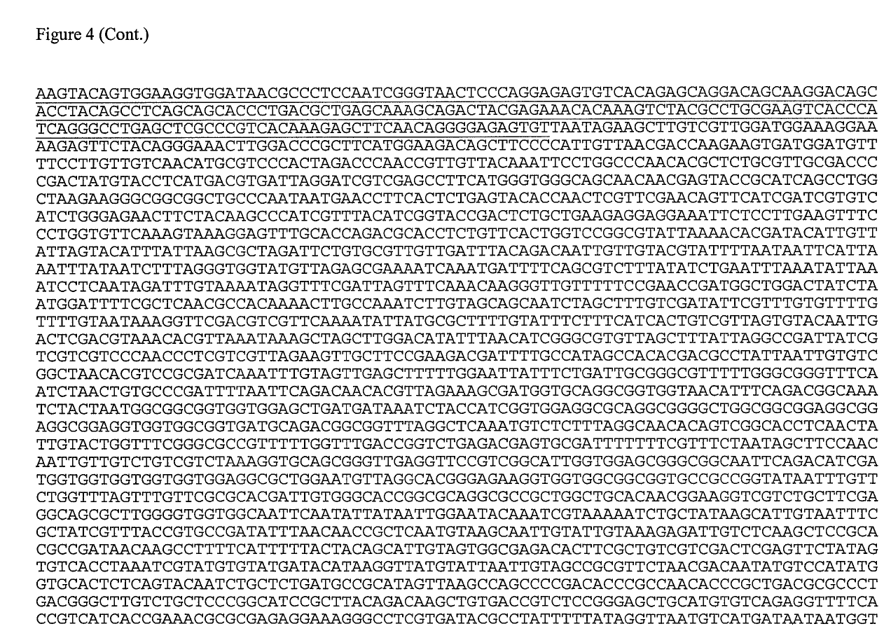

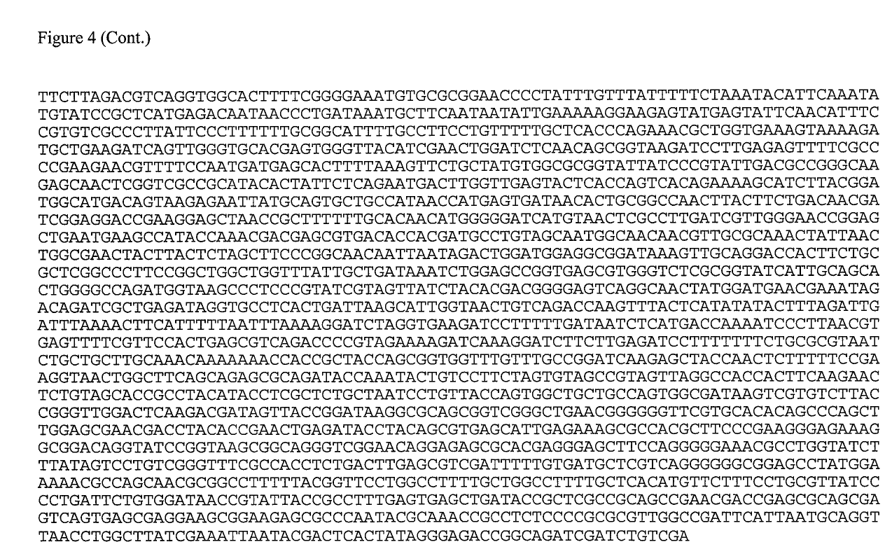

The base vector for the pTRABacHuLC_{κ}HC_{γ1} and pTRABacHuLC_{λ}HC_{γ1} constructs was p2Bac (FIG. 2, SEQ ID NO:5, Invitrogen, Carlsbad, Calif.). Two secretory signal sequences were cloned into this base vector, and the first intermediate baculovirus expression vector p2BacM was created. In general, the vector p2Bac was first modified utilizing complimentary oligonucleotides encoding the amino terminal domain of the honey bee melittin secretory signal sequence positioned to be under transcriptional control of the baculoviral AcNPV P10 promoter. For melittin sequence cloning, 2 μg p2Bac was digested with Not I and Spe I for 4 hours at 37° C. The linear vector was purified following electrophoresis through a 1% agarose gel using Qiaex II resin (Qiagen, Chatsworth, Calif.). The purified DNA was then eluted with 50 μl water and the DNA concentration was determined. One μg each of primers Me1S/N (SEQ ID NO:16) and Me1N/S (SEQ ID NO:17) were mixed in 10 μl digestion buffer M (Roche Molecular Biochemicals, Indianapolis, Ind.), and heated to 70° C. for 5 min, then cooled to room temperature to anneal complimentary primers. Ten percent of the annealed primers was digested in 20 μl reaction with Not I and Spe I for 4 hours at 37° C., and the digested primers were purified following electrophoresis through a 15% polyacrylamide gel with Qiaex H resin, and the concentration of the DNA for annealed primers was determined. The DNAs of p2Bac vector and annealed melittin fragment were ligated at 1:10 vector to insert ratio. The ligation product was transformed using competent XL1-Blue E. coli (Stratagene, San Diego, Calif.) and plated on a LB-carbenicillin agar plate for overnight growing at 37° C. Miniprep colonies were prepared by standard protocols, and the plasmids were sequenced to check the construction. The resulting vector p2BacM contained the melittin secretory signal sequence.

The p2BacM vector was further modified similarly to encode for the amino terminal domain of the human placental alkaline phosphatase secretory signal sequence under transcriptional control of the AcNPV polyhedron promoter, creating a second intermediate baculovirus expression vector p2BacMA. The procedure used to introduce the alkaline phosphatase sequence was generally cloned as follows: 2 μg p2BacM plasmid was digested with Bam HI and Eco RI, the linear vector was gel purified from agarose gel with Qiaex II resin and eluted in 50 μl water. The DNA concentration of the vector was determined. One μg each of primers APB/E (SEQ ID NO: 18) and APE/B (SEQ ID NO:19) were mixed in 10 μl digestion buffer M, and heated to 70° C. for 5 min and then cooled down to room temperature to anneal complimentary primers. Ten percent of the annealed primers was digested in a 20 μl reaction with Bam HI and Eco RI for 4 hours at 37° C. The digested primers were then purified from 15% polyacrylamide gel with Qiaex II resin. The DNA concentration of the digested primers was also determined. The linear p2BacM vector and alkaline phosphatase fragment were then ligated at 1:10 vector to insert ratio, and the ligation product was transformed using competent XL1-Blue E. coli and plated on a LB-carbenicillin agar plate for overnight growing at 37° C. Miniprep colonies were prepared and the plasmids were sequenced to check the construction. The resulting intermediate vector p2BacMA would contain a secretory signal sequence for a human placental alkaline phosphatase. The p2BacMA plasmid was further transformed into SCS-110 E. coli strain (Stratagene) lacking dcm methylase activity for subsequent cloning of the κ constant region into methyl-sensitive Stu I site.

Amplification and Cloning of Constant Regions of IgG_{γ1} and Light Chains:

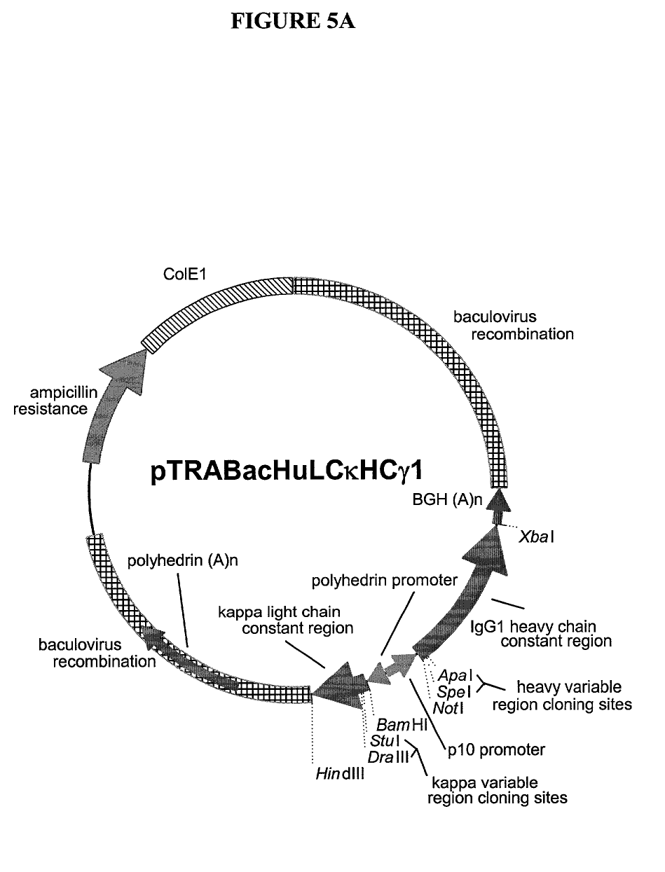

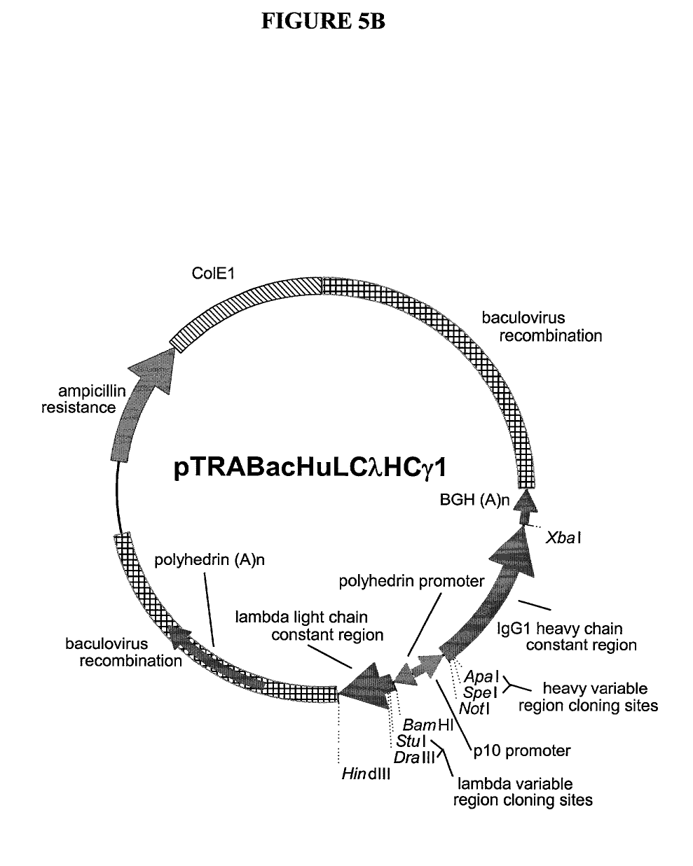

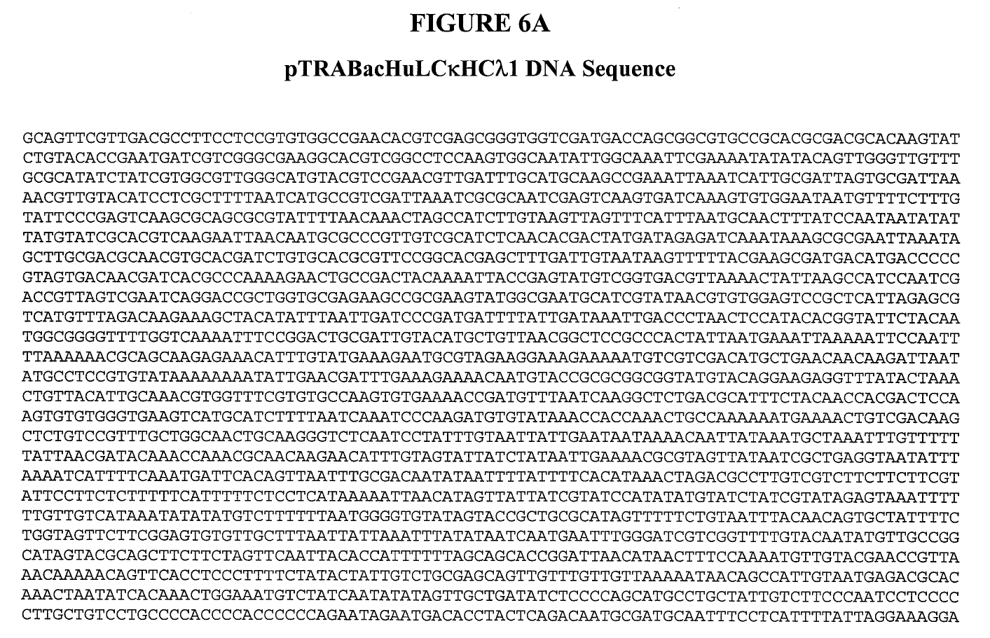

The human kappa (κ) constant and the human IgG_{γ1} constant domains of human monoclonal antibody 9F12 were PCR amplified from RNA extracted from the human cell line 9F12 (ATCC#HB8177). The κ constant region was cloned behind the alkaline phosphatase signal sequence. The IgG_{γ1} constant region was inserted downstream from the melittin secretory signal sequence thus creating the vector (pTRABacHuLC_{κ}HC_{γ1}, FIG. 5a). A vector containing the human lambda (λ) light chain constant region (pTRABacHuLC_{λ}HC_{γ1}, FIG. 5b) was produced by replacing the κ light chain constant region with a λ light chain constant region. The light chains were isolated by RT-PCR from a chronic lymphocytic leukemia cellular RNA preparation. The detailed description of the cloning procedures are as follows.

Amplification of 9F12 κ and IgG_{γ1} Constant Region Fragments:

Total RNA from 9F12 cells (ATCC#HB8177) was extracted using the RNeasy Kit (Qiagen) as per the manufacturer's instruction. A single stranded cDNA was synthesized using SuperScript reverse transcriptase (GIBCO BRL, Rockville, Md.) with oligo(dT) primers. One twentieth of the synthesized single strand cDNA was amplified in 100 μl PCR reactions with Expand High Fidelity Taq (Roche) using κ and IgG_{γ1} specific oligonucleotides (SEQ ID NO:22 plus SEQ ID NO:23 and SEQ ID NO:20 plus SEQ ID NO:21, respectively). The fragments from amplified 9F12 immunoglobulin were purified from 1.5% SeaKem agarose with Qiaex II resin and eluted with 50 μl water. The DNA concentrations for the fragments were determined. The purified 9F12 immunoglobulin fragments were ligated separately into the TA-II (Invitrogen) PCR cloning vector. The ligation products were transformed using competent XLI-Blue E. coli and plated on a LB-carbenicillin agar plate for overnight growing at 37° C. Miniprep colonies were prepared and the plasmid DNA was sequenced.

Insertion of the 9F12 κ Constant Region into the Expression Vector:

For κ constant domain, 5 μg plasmid DNA containing a κ constant region and 2 μg of DNA for the vector p2BacMA purified from SCS 110 E. coli were digested with Stu I and Hind III. A 320 bp fragment containing κ constant region and a 7.1 kb fragment containing p2BacMA vector were gel purified with Quiex II and eluted in 50 μl water. The DNA concentrations for both fragments were determined. The purified fragments were then ligated with Rapid Ligation Kit (Roche). The ligation products were transformed using competent XL1-Blue E. coli and plated on a LB-carbenicillin agar plate for overnight growing at 37° C. Miniprep bacterial colonies were prepared and the recombinant DNA was sequenced to verify proper κ constant region insertion. The resulting plasmid vector was pTRABacLC_{κ}.

Addition of the IgG_{γ1} Constant Domain to the Vector:

The IgG_{γ1} constant domain was added to the vector by first digesting 5 μg of plasmid DNA containing IgG_{γ1} constant region and 2 μg plasmid DNA for the vector pTRABacLC_{κ} with Spe I and Xba I. A 1 kb fragment of IgG_{γ1} constant region and a 7.4 kb fragment of pTRABacLC_{κ} vector were gel purified from agarose plugs with Quiex II and eluted in 50 μl water. The DNA concentrations for both fragments were determined. The purified fragments were then ligated with Rapid Ligation Kit (Roche). The ligation products were transformed using competent XL1-Blue E. coli and plated on a LB-carbenicillin agar plate for overnight growing at 37° C. Miniprep colonies were prepared and the ligation and orientation of the IgG^{γ1} insertion were determined by restriction analysis and sequencing of the restriction sites. The resulting recombinant vector was pTRABacHuLC_{κ}HC_{γ1}.

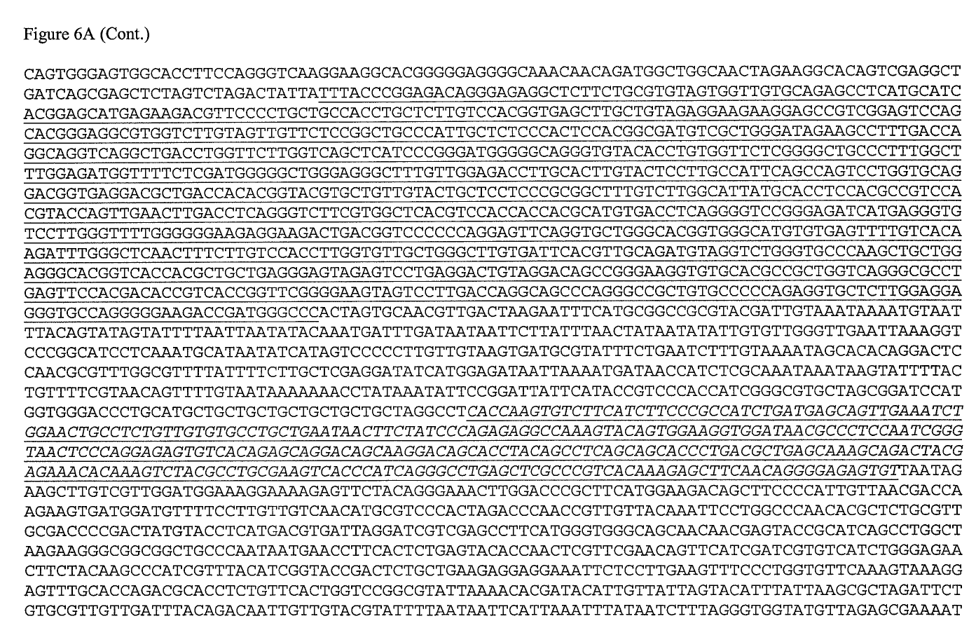

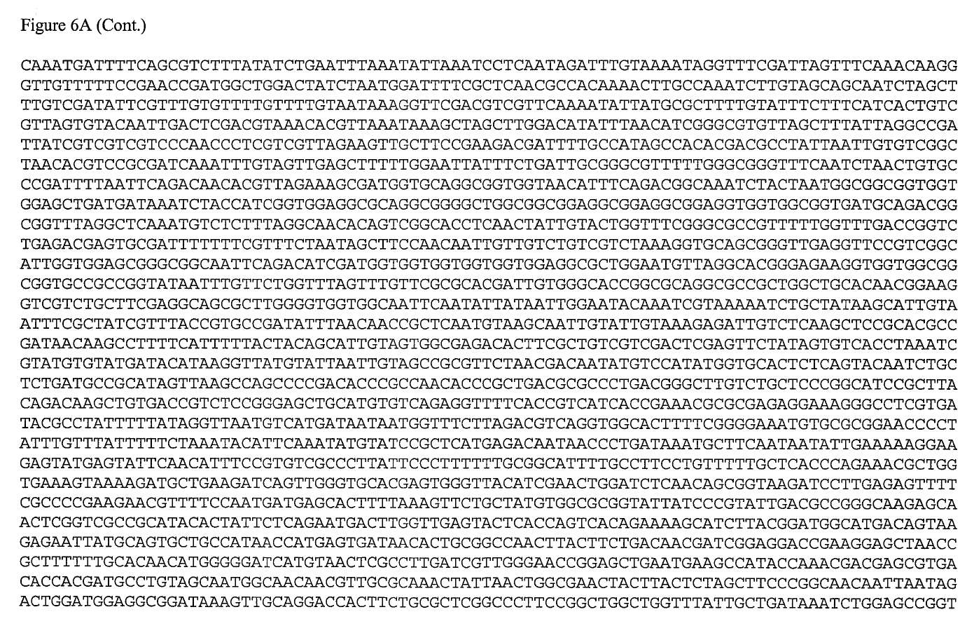

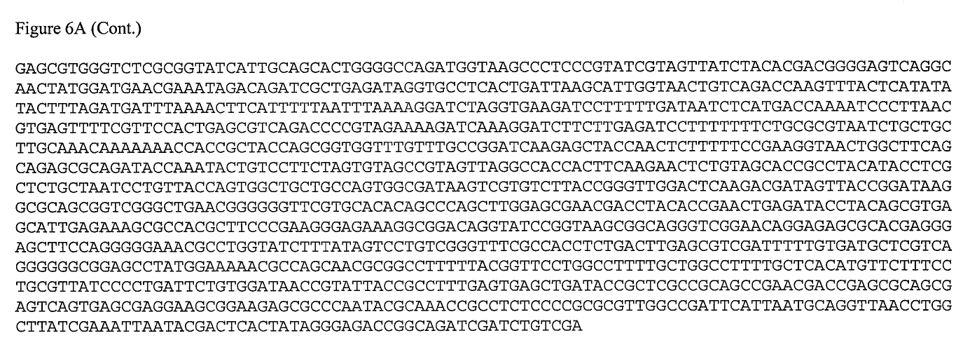

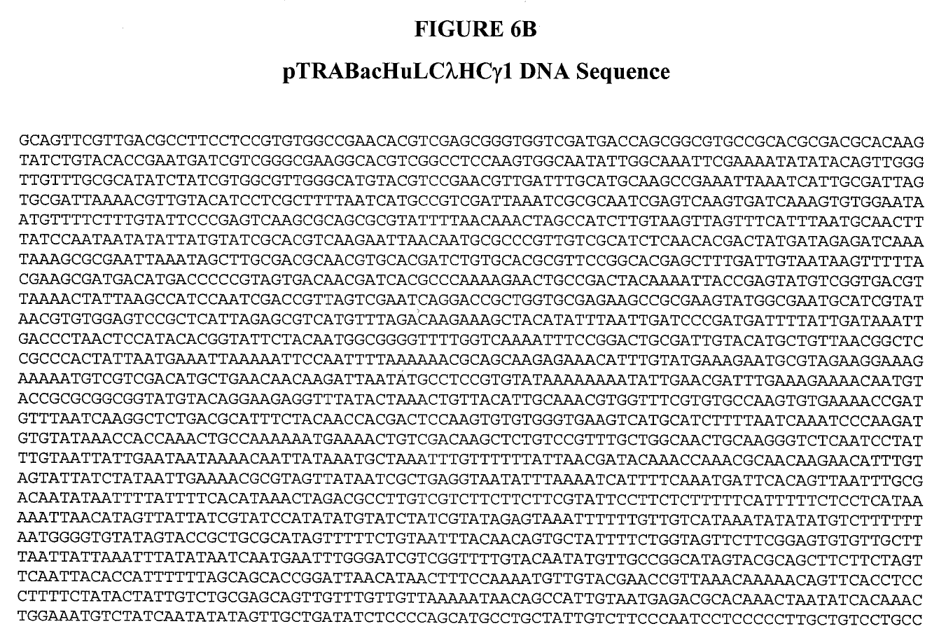

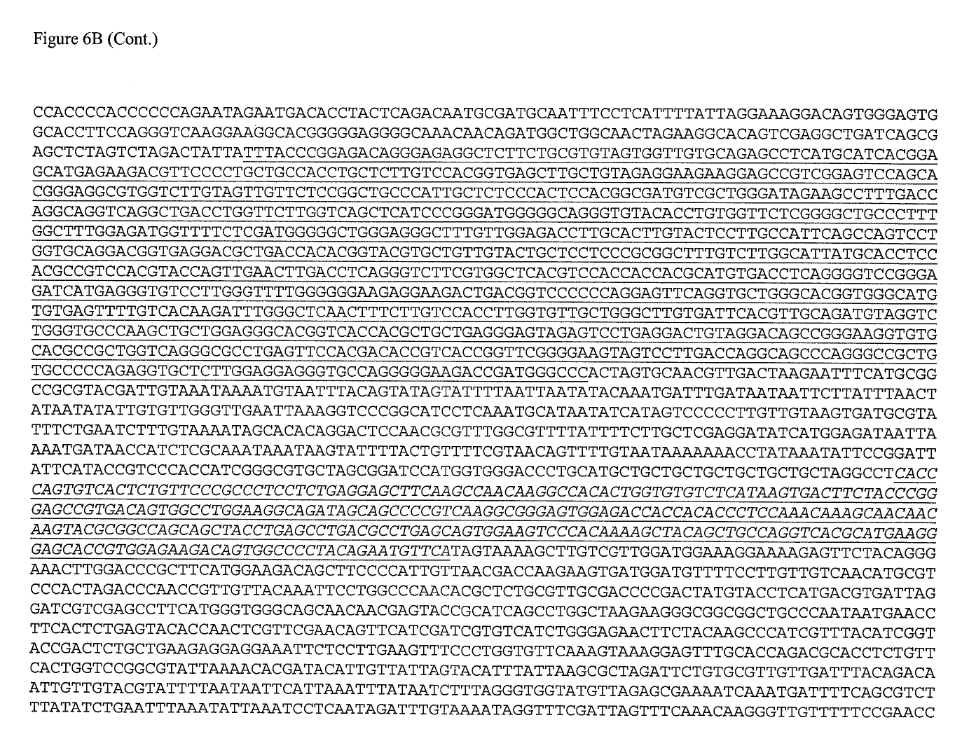

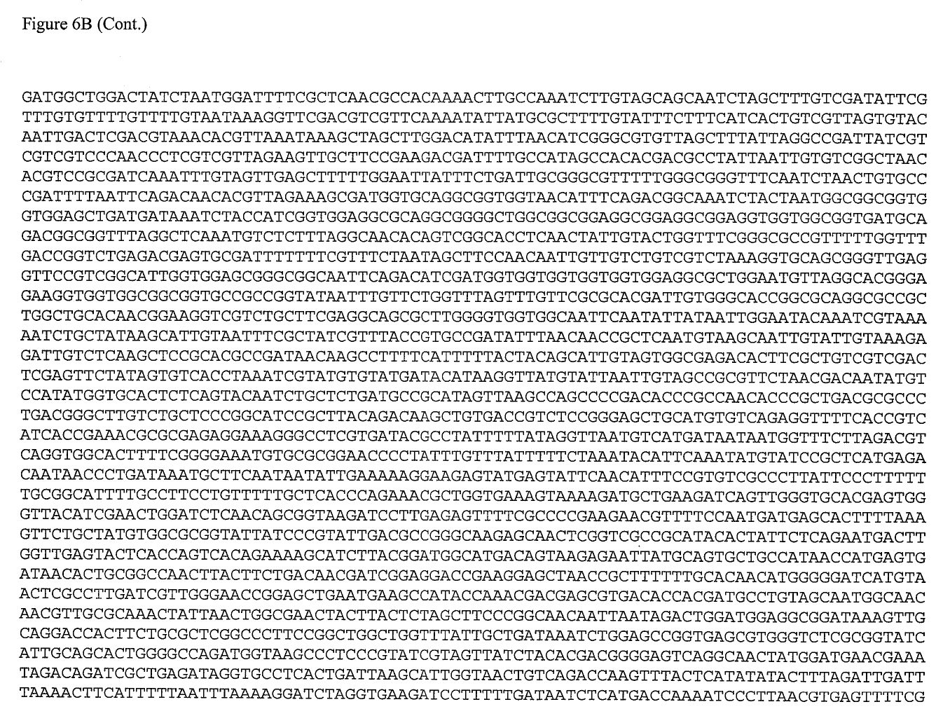

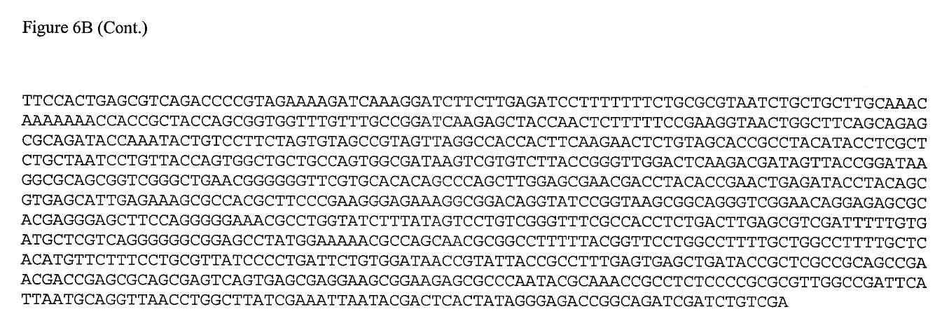

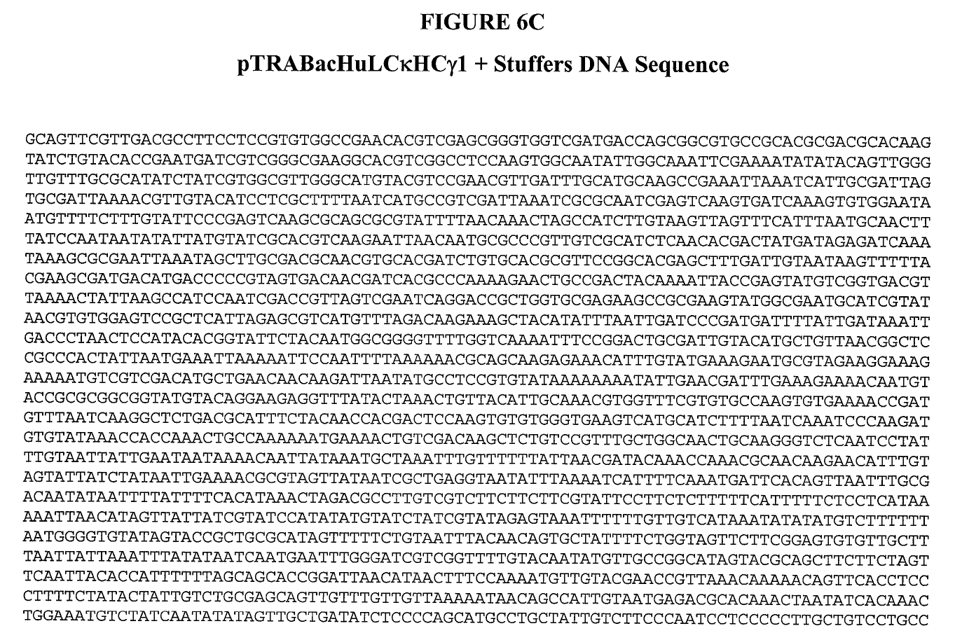

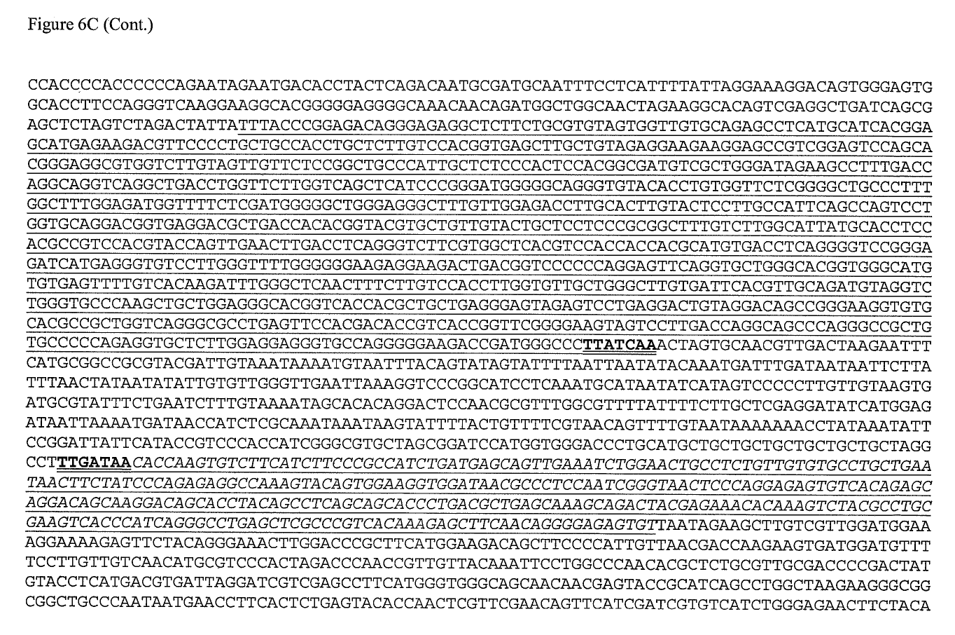

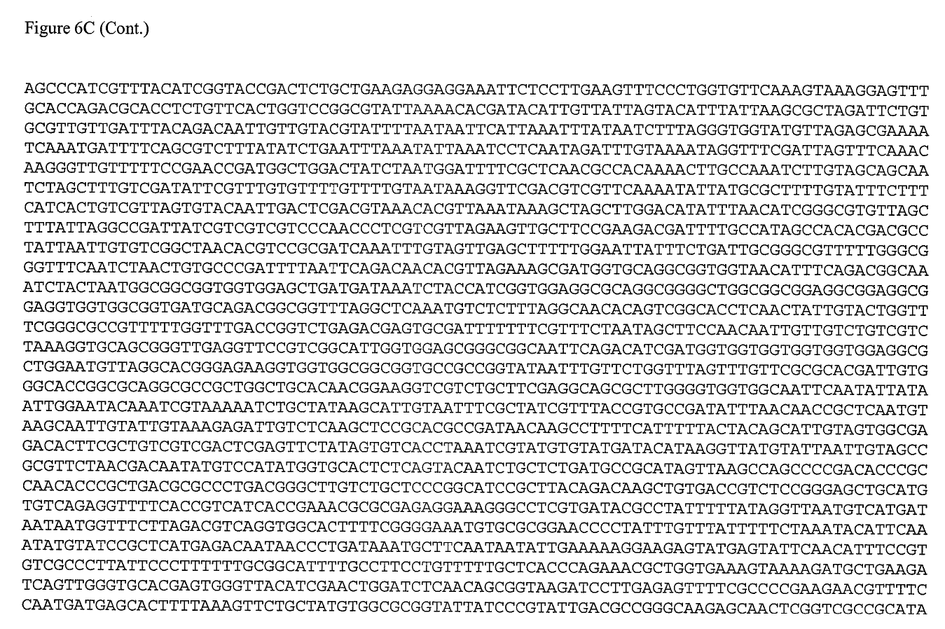

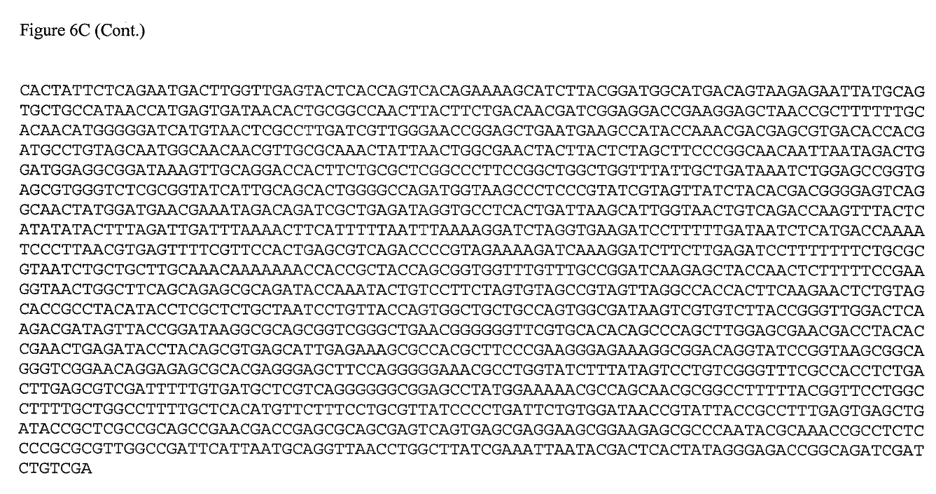

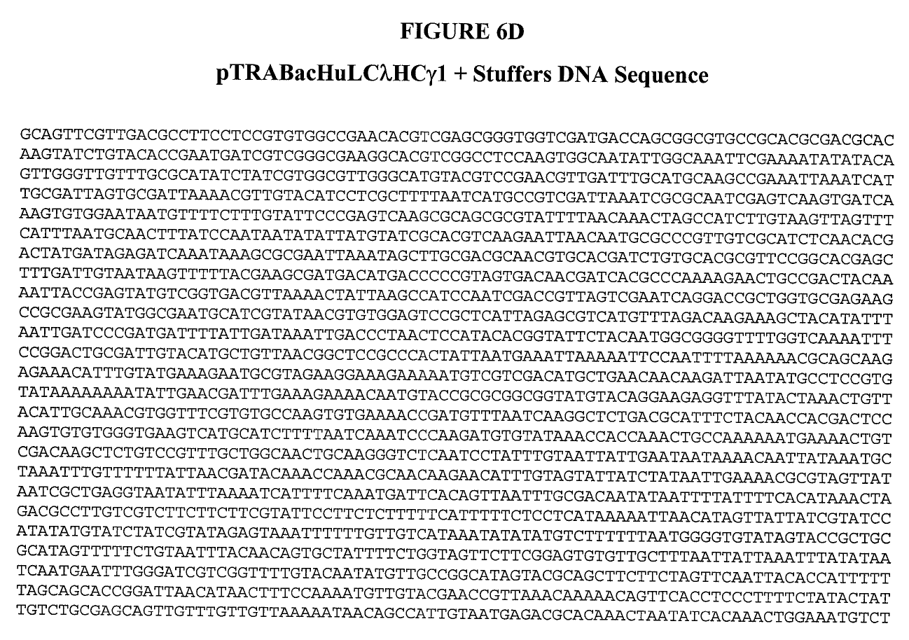

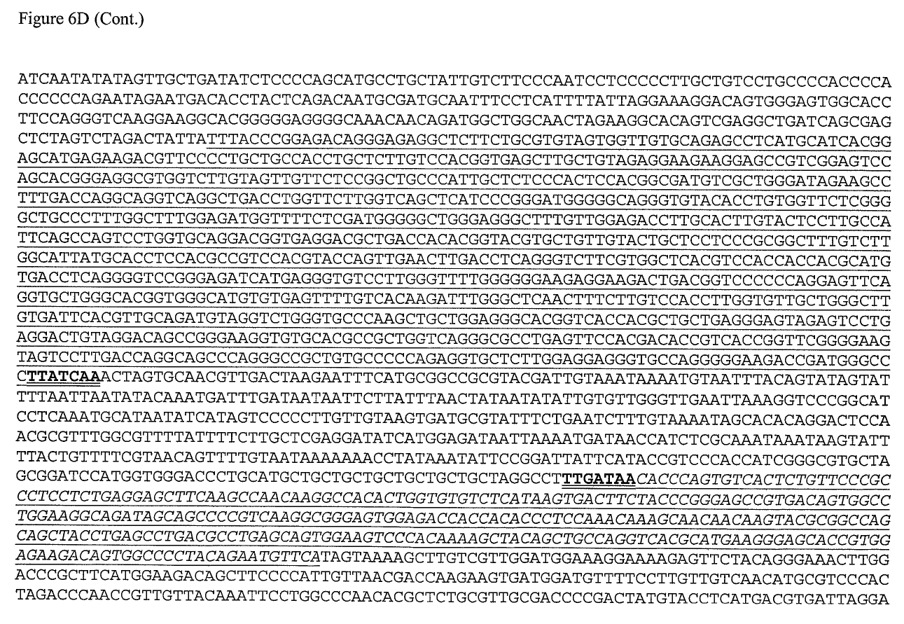

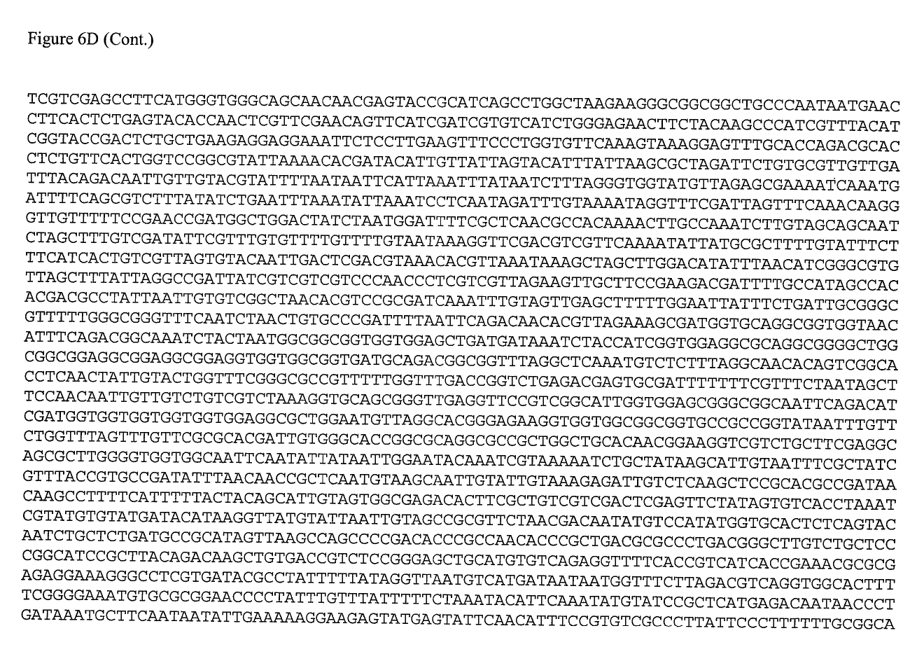

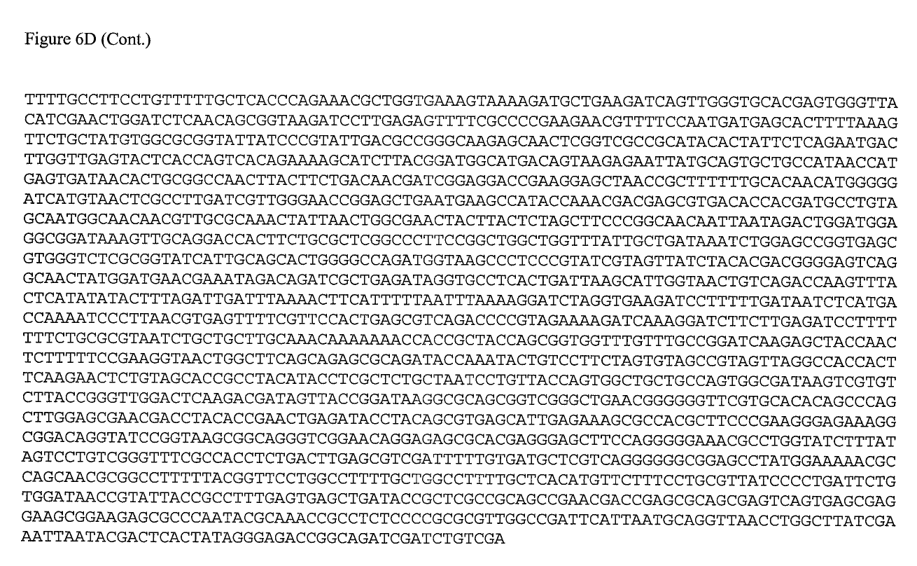

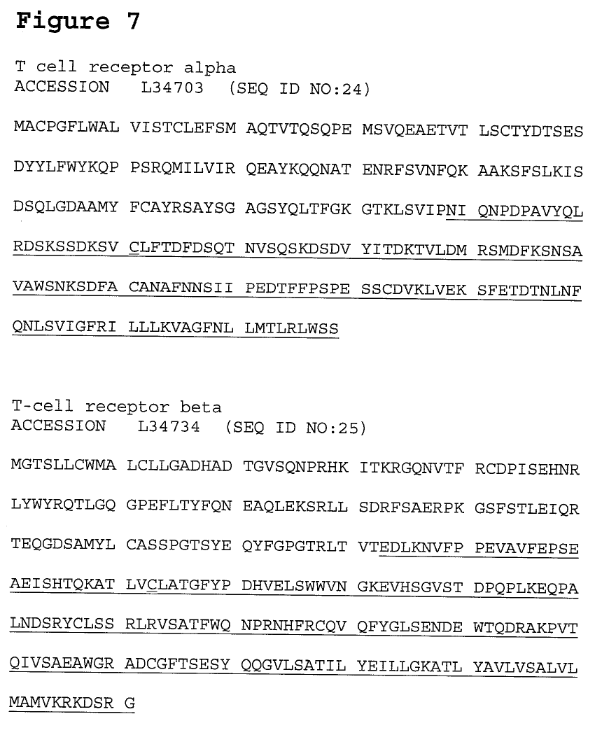

This plasmid, pTRABacHuLC_{κ}HC_{γ1}, was further refined to add translational stop codons between the melittin secretory sequence, and the C_{γ1} region sequence and the alkaline phosphatase secretory sequence and the C_{κ} region sequence, respectively. To accomplish these modifications, the pTRABacHuLC_{κ}HC_{γ1} vector was linearized following digestion with Spe I+Apa I. The linearized vector was then ligated with annealed complimentary primers γ1-stuff 1 (SEQ ID NO:53) and γ1-stuff 1′ (SEQ ID NO:54) to introduce the in-frame stop codons. The vector resulting from this modification was subsequently linearized following digestion with Stu I (AGGCCT)+Dra III (CACnnnGTG) and then ligated with annealed complimentary primers κ-stuff 1 (SEQ ID NO:55) and κ-stuff 1′ (SEQ ID NO:52) to introduce the in-frame stop codons. The net effect of these modifications are indicated in the sequences shown in FIGS. 6C & 6D, respectively. (The added sequences are highlighted by a double underline and bold.)

Addition of the λ Constant Region to the Vectors:

Total RNA from purified peripheral blood lymphocytes (PBL) obtained from a chronic lymphocytic leukemia (CLL) patient displaying a λ light chain idiotype was extracted using the RNeasy kit (Qiagen).

Approximately 2.0 μg total RNA was used as template for first strand cDNA synthesis using the SuperScript Preamplification System (Gibco BRL) according to manufacturer's recommendation. Oligo(dT) was used for priming. One twentieth of the synthesized single stranded cDNA was amplified in a PCR reaction using an upstream primer identical to a portion of the Vλ signal sequence (TTGCTTACTG CACAGGATCC GTG; SEQ ID NO:47) and a downstream primer (TGCCGTCGGC AGGAGGTATT TCATTATGAC TGTCTCCTTG CTATTATGAA CATTCTGTAG GGGCCA; SEQ ID NO:48) complimentary to the last several codons of the λ constant region as well as a portion of the 3′ untranslated region. The PCR products were cloned into the pCRII vector (Invitrogen) and sequenced to confirm identity. A plasmid containing the correct λ constant region sequence was chosen as a template for a second PCR. In this reaction a sense oligonucleotide, Cλ-5′ (SEQ ID NO:49), containing an engineered Dra III restriction site, corresponding the sequence in the λ constant region immediately downstream of Jλ and a Hind III containing antisense oligonucleotide primer, Cλ-3′ (SEQ ID NO:50) spanning the STOP codon immediately following the λ constant region were utilized. The resulting PCR product was cloned into the pCR2.1-TOPO vector and sequenced. A fragment containing the λ constant region sequence was released upon Hind III restriction from some of the plasmids, depending on orientation of the insert. This restriction fragment was gel isolated and cloned into pTRABacHuLCκHCγ1 (FIG. 5A), following linearization following Hind III digestion, generating an intermediate plasmid containing both the λ and κ constant regions. Restriction of this plasmid with Stu I and Dra III resulted in the removal of the κ sequences. This linearized plasmid was then ligated with annealed complimentary primers λ-stuff 1 (SEQ ID NO:5 1) and λ-stuff 1′ to generate the final version of pTRABacHuLCλHCγ1 (FIG. 5B).

A list of all oligonucleotide primers used in the construction of pTRABacHuLC_{κ}HC_{γ1} and/or pTRABacHuLC_{λ}HC_{γ1} can be found in Tables 2 and 3 below.

Using either pTRABacHuLC_{κ}HC_{γ1} or pTRABacHuLC_{λ}HC_{γ1} it is possible to insert genes for any V_{α} or V_{β} chain containing the unique cloning sequences Stu I and Dra III between the κ or λ constant region and the alkaline phosphatase signal sequence, and genes for any V_{β} or V_{α} chain containing the unique cloning sequences Spe I and Apa I between the IgG_{γ1} constant region and melittin secretory signal sequence. The resulting expression vector could then be utilized for transduction into Spodoptera frugiperda (Sf-9) insect cells to produce recombinant budded baculovirus. The recombinant baculovirus was then serially amplified in Sf-9 cells to produce a high titer recombinant baculovirus stock. This high titer recombinant baculovirus stock was then used to infect Trichoplusia ni (High-5) cells for subsequent chimeric TCR/Ig protein production.

TABLE 2
PRIMER SEQUENCES

| PRIMER NAME | PRIMER SEQUENCE (5′ 3′) | |
|---|---|---|
| Cλ Downstream | TGCCGTCGGCAGGAGGTATTTCATTATGACTGTCT | SEQ ID NO:48 |
| | CCTTGCTATTATGAACATTCTGTAGGGGCCA | |
| Cλ - 5′ | GTCAGCCCAAGGCTGCACCCAGTGTCACTCTGTTCC | SEQ ID NO:49 |
| Cλ - 3′ | CGTATCAAGCTTTTACTATGAACATTCTGTAGGGGCCAC | SEQ ID NO:50 |
| λ-stuff 1 | CCTTTGATAACACCCA | SEQ ID NO:51 |
| λ-stuff 1′ | GTGTTATCAAAGG | SEQ ID NO:52 |
| γ1-stuff 1 | 5′-CTAGTTTGATAAGGGCC-3′ | SEQ ID NO:53 |
| γ1-stuff 1′ | 5′-CTTATCAAA-3′ | SEQ ID NO:54 |
| κ-stuff 1 | 5′-CCTTTGATAACACCAA-3′ | SEQ ID NO:55 |
| κ-stuff 1′ | 5′- -3′ | SEQ ID NO:56 |

### Example 3

### Insertion of Genes for Patient-Derived Idiotype V_{α} and/or V_{α} Chains into an Expression Vector

After the tumor derived sequences for V_{α} and/or V_{β} chains are isolated as described above, oligonucleotide primers containing the terminal 40 nucleotides of the melittin leader peptide (for V_{β} chain cloning) (SEQ ID NO:8—ACTAGTTTTT ATGGTCGTGT ACATTTCTTA CATCTATGCG), the terminal 31 nucleotides of the alkaline phosphatase leader peptide (for V_{α} chain cloning) (SEQ ID NO:9—AGGCCTGAGG CTACAGCTCT CCCTGGGC), and the first 20 nucleotides of the respective V_{α} or V_{β} genes determined from the analysis described supra are prepared. Reverse oligonucleotide primers complementary to base pairs 4 to 36 of CA (CA/IgK; SEQ ID NO:15) and base pairs 6 to 35 of CB (CB/IgG₁; SEQ ID NO:14) from the α or β chain constant region. Recombinant plasmids identified previously as having the clonal V_{α} or V_{β} sequences are used as templates for a second round of PCR. Cycling conditions were as described supra.

V_{α} Region Insertion into an Expression Vector:

PCR derived DNA fragments amplified from sequence verified plasmid preparations are digested with the restriction enzymes Stu I and Dra III and then separated on agarose gels. The predicted approximately 360 bp fragment containing the V_{α} gene is eluted and inserted into the appropriate baculovirus immunoglobulin expression transfer vector. Basically, a PCR derived V_{α} product and 2 μg of the corresponding pTRABacHuLC_{κ}HC_{γ1} or pTRABacHuLC_{λ}HC_{γ1} cassette vector is digested with Stu I and Dra III. The 360 bp DNA fragment from the patient derived Vₐ chain and the 8.4 kb fragment for the linear pTRABacHuLC_{κ}HC_{γ1} or pTRABacHuLC_{λ}HC_{γ1} vector is purified from agarose gel plugs with Qiaex II resin and eluted in 50 μl water. The DNA concentrations for both fragments are determined and then the fragments are ligated using Rapid Ligation kit (Roche). The ligation products are transformed using competent XL1-Blue E. coli and plated on a LB-carbenicillin agar plate for overnight growing at 37° C. Miniprep colonies are prepared and the recombinant DNA plasmids are verified by restriction analysis and sequencing. The resulting vector is designated pTRABacV_{α}HuLC_{κ}HC_{γ1} or pTRABacV_{α}HuLC_{λ}HC_{γ1}.

V_{β}Chain Insertion into an Expression Vector:

PCR derived DNA fragments amplified from sequence verified plasmid preparations are digested with the restriction enzymes Spe I and Apa 1. The excised approximately 360 bp fragment containing the V_{β} gene is gel purified and inserted into the appropriate baculovirus immunoglobulin expression transfer vector, pTRABacV_{α}HuLC_{κ}HC_{γ1} or pTRABacV_{α}HuLC_{λ}HC_{γ1} containing the associated V_{α} gene or into pTRABacHuLC_{κ}HC_{γ1} without an associated V_{α} gene. Basically, PCR derived V_{β} product and 2 μg DNA of pTRABacVₐHuLC_{κ}HC_{γ1}, pTRABacV_{α}HuLC₈₀ HC_{γ1}, pTRABacHuLC_{κ}HC_{γ1} vector is digested with Spe I and ApaI. A 360 bp fragment of V_{β} chain and an 8.8 kb fragment of the pTRABacV_{α}HC_{γ1}, pTRABacV_{α}HuLC_{κ}HC_{γ1}, or pTRABac V_{α}HuLC_{λ}HC_{γ}1 vector or the 8.4 kb fragment of the pTRABacHuLC_{κ}HC_{γ1} vector is gel purified from an agarose gel plugs with Qiaex II resin and eluted in 50 μl water. The DNA concentrations for both fragments is determined and the fragments are ligated using Rapid Ligation kit (Roche). The ligation products are transformed using competent XL1-Blue E. coli and plated on a LB-carbenicillin agar plate for overnight growing at 37° C. Miniprep colonies are prepared and the recombinant DNA plasmids were sequenced. The resulting vector is designated pTRABacV_{α}HuLC_{κ}V_{β}HC_{γ1}, or pTRABacV_{α}HuLC_{λ}V_{β}C_{γ}1 or pTRABacHuLC_{κ}V_{β}HC_{γ1}.

TABLE 3
Primer sequences used for construction of pTRABacHulC_{κ}HC_{γl} and
pTRABacHulC_{λ}HC_{γl} baculovirus transfer vectors.

| PRIMER NAME | PRIMER SEQUENCE (5′ 3′) | |
|---|---|---|
| 1. Melittin N-terminus | ACTAGTGCAACGTTGACTAAGAATTTCATGCGGCCGC | (SEQ ID NO:16) |
| (MelS/N and MelN/S) | GCGGCCGCATGAAATTCTTAGTCAACGTTGCACTAGT | (SEQ ID NO:17) |
| 2. Human Placental | GCGGATCCATGGTGGGACCCTGCATGCTGCTGCTGCTG | (SEQ ID NO:18) |
| Alkaline Phosphatase | CTGCTGCTAGGCCTGGAATTCC | |
| N-terminus (APB/E and | GGAATTCCAGGCCTAGCAGCAGCAGCAGCAGCAGCATG | SEQ ID NO:19) |
| APE/B) | CAGGGTCCCACCATGGATCCGC | |
| 3. IgG_{γl} Heavy Chain | TGTGACTAGTATGTATCGGCCCATCGGTCTTCCCCCT | (SEQ ID NO:20) |
| Constant: Upstream | | |
| Downstream | TTTCTAGACTATTATTTACCCGAGACAGGGAGAG | (SEQ ID NO:21) |
| 4. Kappa Light Chain | CTAGGCCTATGTATCACCAAGTGTCTTCATCTTCCCGCCATCT | (SEQ ID NO:22) |
| Constant: Upstream | | |
| Downstream | CCCAAGCTTCTATTAACACTCTCCCCTGTTGAAGCT | (SEQ ID NO:23) |

### Example 4

### Transfection of Insect Cell Lines with Baculovirus Expression Vectors Containing V_{α} and/or V_{β} Chain Idiotype and Production of Recombinant Chimeric Proteins

Insect Cell Growth:

Two established insect cell lines (Sf9 and High-5) were transfected with modified baculoviral vectors to produce recombinant chimeric V_{H}/immunoglobulin and/or V_{L}/immunoglobulin proteins. All insect cells were grown at 28° C. in ESF-921 Serum Free Insect Media (Expression Systems LLP) containing 50 μg/L gentamycin in disposable sterile vented shaker flasks (Coming), at 140-150 rpm, with no more than 50% liquid volume. Cells were passaged every 2 to 3 days. Frozen cells were thawed (Cryo-preservation media: 10% DMSO, 40% ESF-921 medium, 50% High-5 conditioned media) from a working cell bank for each lot of product or every six weeks to assure a continuous stock of exponentially growing cells that was not retractile to infection by baculovirus.

Sf9 Cell Transfection and Recombination Assay:

The modified baculovirus expression vectors containing genes for V_{α} and/or V_{β} regions and genes encoding immunoglobulin heavy and/or light chain constant regions were co-transfected into Sf9 cells using the BacVector-3000 transfection kit (Invitrogen). Ten individual plaques are picked from agarose overlays. Virus from isolated plaques are used to infect T-25 flasks seeded with Sf-9 cells at 50% confluency in 5 ml ESF-921 media. Clonal viral isolates amplified in T-25 flasks are tested by PCR, using two primers (SEQ ID NO:37—TTTACTGTTT TCGTAACAGT TTTG) and (SEQ ID NO:38—GGTCGTTAAC AATGGGGAAG CTG) to assure clonality of the isolated plaques and that there was no wild type virus contamination. In general, 200 ng recombinant transfer vector plasmid was co-transfected with triple-cut Bac-Vector-3000 as described in the Bac Vector manual (Novagen) using the Eufectin lipid reagent supplied. This transfection mixture was subjected to serially 5-fold dilutions. One hundred microliter aliquots were plated in 60 mm tissue culture dishes containing 2.5×10⁶ adherent Sf9 cells. After 1 hour, cells were overlaid with 4 ml of a 1% agarose solution in ESF-921 culture medium. Ten individual clones were picked from the transfected cells grown in agarose overlays after staining for live cells using Neutral Red (Sigma, St. Louis, Mo.) at t=144 hours post transfection. Virus was eluted from plaque plugs overnight in 1 ml ESF-921 media. T-25 flasks were seeded with Sf-9 cells at 50% confluency in 5 ml ESF-921 media, and infected with 0.5 ml of eluted clonal virus. Ninety-six hours post infection, 0.5 ml media was removed from T-25 flasks; the cells were removed by centrifugation and the supernatant was assayed for immunoglobulin activity by dot blotting on nitrocellulose. The absence of wild type virus was also tested by PCR as follows.

Infectious supernatant (10 μl) containing recombinant baculovirus was added to 90 μl of lysis buffer containing 10 mM Tris pH 8.3, 50 mM KCl, 0.1 mg/ml gelatin, 0.45% Nonidet P-40, and 0.45% Tween-20, containing 6 μg Proteinase-K. The mixture was heated for 1 hour at 60° C. and the Proteinase-K was denatured by incubation at 95° C. for 10 min. Twenty five μl of the heated mixture was removed to a fresh PCR tube after cooling, and another 25 μl of the mixture containing 10 mM Tris pH 8.3, 50 mM KCl, 0.1 mg/ml gelatin, 0.45% NP-40, 0.45% Tween-20, 400 μM each dNTP, 5 mM MgCl₂, 50 pM each PCR primer (final), and 2.5 U Taq polymerase (Roche) was added. The viral DNA was amplified for 40 cycles at: 92° C. for 1 min., followed by 58° C. for 1 min. and 72° C. for 1 min. The recombinant baculovirus primers PH forward (SEQ ID NO:37) and PH reverse (SEQ ID NO:38) were used to amplify the polyhedron locus expressing the portion of the T cell receptor gene. PCR products were analyzed following electrophoresis through an agarose gel. Recombinant baculovirus would amplify a 1300 bp fragment, while wild type baculovirus would produce a ˜800 bp fragment with these primer sets. Recombinant virus contaminated with wild type virus would amplify both fragment sizes.

Preparation of High Titer Viral Stocks in Sf-9 Insect Cells:

Two ml from a T-25 primary culture is transferred to a T-75 flask containing Sf-9 cells at 50% confluency in 10 ml ESF-921 media, and cells are grown for 120 hours at 28° C. Five ml of secondary T-75 cultures is transferred to a 150 ml shaker flask containing 50 ml of Sf-9 cells at 2×10⁶ cells/ml, and cells are grown for 120 hours at 28° C. Twenty-five ml from 150 ml shaker flask are transferred into 500 ml of Sf-9 cells at 2×10⁶ cells/ml in a IL shaker flask, and grown at 28° C. When the cultures reached 20%, viable cells are scored by trypan blue staining (˜t=120-144 hours post infection), the viral culture is harvested by centrifugation at 3000× g, distributed into 50 ml sterile tubes, and half of the tubes are stored at 4° C. with the rest at −80° C. This harvested 500 ml high titer (>1×108 pfu/ml) viral stock is then used to infect High-5 insect cells for chimeric protein production. Viral titers (pfu/ml) are determined using a Baculovirus Rapid Titer Kit (Clontech, Palo Alto, Calif.).

Production of TCR/Ig Chimeric Protein in High-5 Insect Cells:

High-5 insect cells (BTI-TN-5B1-4) secreted higher levels (2-20×) of recombinant immunoglobulin compared to Sf-9 cells, and are the cell line of choice for TCR/Ig chimeric protein production. Early log phase High-5 cells (1.0-2.0×10⁶ cells/ml) are seeded in 1 liter disposable culture flasks with vented closures at 5×10⁵ cells/ml in ESF921 Media (Expression Systems LLP). The flasks are shaken at 140-150 rpm at 28° C., and the volume of media in the flasks is adjusted over time to no greater than 500 ml. When the cell densities reached 1.5-2.5 cell/mi in 500 ml media, the flasks are infected with high titer recombinant baculovirus stock at a multiplicity of infection (MOI) approximating 0.5:1 (pfu:cells). The flasks are then shaken at 140-150 rpm at 28° C. Cell viability is checked daily, and the culture is harvested in the event that the viability did not drop to <50% within 96 hours post-infection.

### Example 5

### Purification of Chimeric Proteins Comprising a V_{α}-Immunoglobulin and a V_{β}-Immunoglobulin

Cells and debris were removed by centrifugation for 60 min. at approximately 5,000× g, followed by filtration through a 0.2μ PES sterile filter unit. Chimeric proteins were purified from cleared tissue culture media by affinity chromatography with a Protein-A High-Trap cartridge (Amersham Pharmacia, Piscataway, N.J.), followed by precipitation in 50% saturated ammonium sulfate. The purified chimeric proteins were size separated and buffer exchanged into PBS by FPLC chromatography. All reagents used for protein purification were of USP biotechnology grade (GenAr, Mallinckrot Baker, Parris, Ky.) and endotoxin tested by the manufacturer. Sterile USP grade water was used to make all buffers and other solutions. Buffers and other solutions were prepared in a biological safety cabinet, and filter sterilized through 0.2 μm PES filter units.

Protein A Sepharose Affinity Purification of the Chimeric Proteins:

Tissue culture medium was removed from growing culture flasks and spun for 60 min. at 5,000× g to sediment cells and debris. The supernatant was sterilized by filtration using a 0.2μ PES filter unit. Tris buffer (1M, pH 7.4) was added to the filtered medium containing V_{H} and/or V_{L}-immunoglobulin chimeric proteins to a final concentration of 20 mM. The buffered tissue culture supernatant was loaded onto a 5 ml HighTrap recombinant Protein A Sepharose affinity cartridge at a flow rate of 1 to 5 ml/min with a peristaltic pump (Amersham Pharmacia) collecting the flow-through in a clean flask. The column was washed with 25 ml PBS (pH 7.4) at 5 m/min. The direction of the flow was reversed and the column was washed with an additional 25 ml PBS. The column was eluted in reverse at 1 ml/min with 0.05 M citric acid (pH 3.5) collecting 1 ml fractions. The pH is immediately adjusted approximately 8.0 by adding approximately 0.3 ml 1M Tris (pH 9.0) to the eluted 1 ml fractions. Other protein columns including but not limited to protein G, protein L, or any proteins that are able to bind to an immunoglobulin binding domain could be used in the same manner.

Ammonium Sulfate Precipitation of the Chimeric Proteins:

The V_{α} and/or V_{β}-Ig containing fractions eluted from Protein A Sepharose are identified by spectrophotometry. The peak fractions are pooled and the volume is determined. An equal volume of saturated ammonium sulfate solution is then added dropwise with mixing. The precipitate was allowed to stand at room temperature for 15 min, then sedimented by centrifugation at 5000× g. The precipitated V_{α} and/or V_{β}-Ig chimeric proteins are resolubilized in sterile water. The V_{α} and/or V_{β}-Ig chimeric proteins are buffer-exchanged into PBS using a High Trap Desalting cartridge (Amersham Pharmacia) followed by final sterile filtration through a 0.2 μl filter.

### Example 6

### Conjugation of Chimeric Proteins with Keyhole Limpet Hemocyanin (KLH)

After purification, the V_{α} and/or V_{β}-Ig chimeric proteins are conjugated to GMP grade KLH (VACMUNE, Biosyn Corporation) via glutaraldehyde crosslinking as follows. At least 5 mg of purified, sterile idiotype chimeric protein is combined with an equal weight of KLH in a sterile 15 ml conical tube, and the final is adjusted to 9 ml in PBS. One ml of 1% glutaraldehyde (25% Grade 1 aqueous solution, Sigma) is added dropwise to a final concentration of 0.1%. The tube is then slowly rocked for 4 hours at room temperature. The conjugate was dialyzed in sterile DispoDialyzers (Spectrum Labs) against 2 liters sterile PBS, with three buffer changes over at least 24 hours in a biological safety hood. The final V_{α} and/or V_{β}-Ig chimeric proteins-KLH conjugates is aseptically removed from the dialysis chambers and transferred into a sterile tube, mixed, then aliquoted into vials. Each vial of final product is labeled with the lot number, patient identifier, vial number and the date it was aliquoted into vials. Ten percent of the final lot is tested for sterility, and a vial is selected and tested for endotoxin.

### Example 7

### Product Tests

DNA Sequence of Baculovirus Containing Production Lot Supernatant:

A 1 ml aliquot of sample of infected insect cell production culture supernatant is harvested and cleared of cellular debris by spinning at 3000 rpm for 5 min in a desktop centrifuge. At least 0.1 ml of this cleared supernatant containing baculovirus particles is combined at a volume ratio of 1 to 9 with lysis buffer (10 mM Tris, pH 8.3, 50 mM KCl, 0.1 mg/ml gelatin, 0.45% Nonidet P-40, and 0.45% Tween-20), subjected to proteolysis with proteinase K (final concentration 60 μg/ml) for 1 h at 60° C., followed by denaturation for 15 min at 95° C. Twenty-five μl of this lysate was then combined with an additional 25 μl of the above lysis buffer containing 400 μM of each dNTP, 5 mM MgCl₂, 25 pmol oligonucleotide (SEQ ID NO:32 and SEQ ID NO:35; or SEQ ID NO:36 and SEQ ID NO:37) (see Table 4), and 2.5 U Taq polymerase (Roche) in order to conduct a PCR reaction. The cycling conditions are: (I) initial denaturation for 2 min at 92° C., (ii) followed by 40 cycles of 1 min each at 92° C., 58° C., and 72° C., and (iii) a final extension of 7 min at 72° C. PCR products are assessed for expected size and quantity by agarose gel electrophoresis. Subsequently, two or more nested primers are used to directly sequence the PCR products. The complete V_{α} and/or V_{β} nucleotide sequences determined using the OpenGene Automated DNA Sequencing System (Visible Genetics) and sequencing analysis software, as described above and compared with the V-gene sequences of the pTRABac(NHL-FV-8786-XXX) vector corresponding to that patient's idiotype.

Analysis of the Chimeric Proteins Using Immunoglobulin Assay of Anti Human IgG ELISA:

Microtiter plate wells are coated with 100 μl of a 3 μg/ml dilution of Goat anti-Human IgG heavy chain specific antibody (Fischer, Pittsburgh, Pa.) in carbonate buffer overnight at 4° C., and washed 2 times with 100 μl TBS (50 mM Tris, 150 mM NaCl, pH 7.5). The wells are then blocked with 200μl TBSB (TBS+1% BSA) for 1 hour at 22° C. A 100 μl diluted sample in 2-fold serial dilutions was added to wells in replicates, and incubated for 1 hour at 22° C. This analysis is repeated with purified Human IgG₁/κ or IgG₁/λ standards (Sigma, St. Louis, Mo.). The wells were washed 4 times with 200 μl TBST (TBS+0.1% Tween 20). The detection antibody, Goat-anti-Human H and L-HRP (Chemicon, Temecula, Calif.) is diluted 1:2000 in TBSB and 100 μl was added to wells for incubation 1 hour 22° C. The wells are washed 6 times with 200 μl TBST. A 100 μl substrate (TMB 1 component, KPL Inc., Gaithersburg, Md.) was added to wells and developed for 30 min and samples are measured by OD₆₅₀ chromatogram. The major protein peak eluted from the Hi Trap Desalting column must correspond to human IgG ELISA activity.

### Example 8

### The Concentration and Purity of the Chimeric Proteins

DNA sequence of V_{α} and/or V_{β} genes in baculovirus in production supernatant must be identical to the DNA sequence in the production vector. The concentration of the chimeric proteins must exceed 0.5 mg/ml based on OD₂₈₀, and must correspond to human IgG ELISA activity.

Table 4 shows a summary of primer sequences used for establishing final product identity.

TABLE 4
Primer Sequences Used for Establishing Final Product Identity.

| PRIMER NAME | PRIMER SEQUENCE (5′ 3′) | |
|---|---|---|
| 1. Human Placental Alkaline | AAATGATAACCATCTCGC | (SEQ ID NO:26) |
| Phosphatase Internal | | |
| 2. Human Placental Alkaline | TTTACTGTTTTCGTAACAGTTTTG | (SEQ ID NO:27) |
| Phosphatase External | | |
| 3. Kappa Light Chain Constant | TTGGAGGGCGTTATCCACCTTC | (SEQ ID NO:28) |
| Antisense | | |
| 4. Kappa Light Chain Constant | CTGTAAATCAACAACGCACAG | (SEQ ID NO:29) |
| Downstream Internal | | |
| 5. Kappa Light Chain Constant | CAACAACGCACAGAATCTAG | (SEQ ID NO:30) |
| Downstream External | | |
| 6. Melittin Internal | GGGACCTTTAATTCAACCCAACAC | (SEQ ID NO:31) |
| 7. Melittin External | AAACGCGTTGGAGTCTTGTGTGC | (SEQ ID NO:32) |
| 8. IgG_{γ1} Heavy Chain Constant | GGAAGTAGTCCTTGACCAGGCAG | (SEQ ID NO:33) |
| Antisense Internal | | |
| 9. IgG_{γ1} Heavy Chain Constant | CTGAGTTCCACGACACCGTCAC | (SEQ ID NO:34) |
| Antisense Middle | | |
| 10. IgG_{γ1} Heavy Chain Constant | TAGAGTCCTGAGGACTGTAGGAC | (SEQ ID NO:35) |
| Antisense External | | |
| 11. Kappa & Lambda Downstream: | 5′-GGTCGTTAACAATGGGGAAGCTG-3′ | (SEQ ID NO:36) |
| 12. PH forward | 5′TTTACTGTTTTCGTAACAGTTTTG3′ | (SEQ ID NO:37) |
| 13. PH reverse | 5′GGTCGTTAACAATGGGGAAGCTG3′ | (SEQ ID NO:38) |
| 14. Lambda Constant Internal | 5′GAAGTCACTTATGAGACACACCAG3′ | (SEQ ID NO:39 |

### Example 9

### Co-Administration of the V_{α} and/or V_{β}-Ig Chimeric Protein and/or Its Conjugates with a Cytokine:

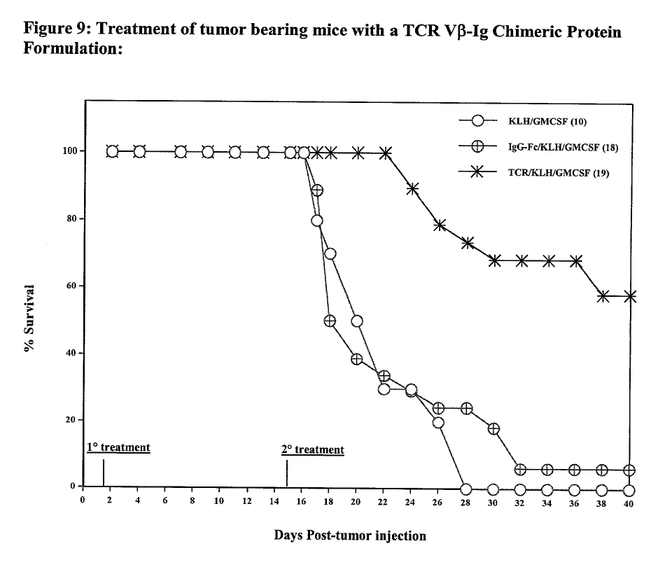

The TCR Vβ12 gene (as described by Van Hall et al., Identification of a Novel Tumor-Specific CTL Epitope Presented by RMA, EL-4, and MBL-2 Lymphomas Reveals Their Common Origin. J. Immunol., 165:869-877; 2000) expressed by the murine T cell lymphoma line, RMA, was cloned as described supra using a 5′ oligonucleotide containing the terminal 40 nucleotides of the melittin leader peptide and the first 20 nucleotides of murine Vβ12 (SEQ. ID. NO:56: TTACTAGTTT TTATGGTCGT GTACATTTCT TACATCTATG CTGACGCTGG AGTTACCCAG A) and a 3′ oligonucleotide complementary to the 5′ end of murine Cb and human IgG1 (SEQ. ID. NO:57: AGGAGACCTT GGGTGGAGTC GGGCCCTTCA GATCCTC). The resulting PCR product (Figure X see last page) was cloned into the Spe I/Apal sites of pTRABacHuLC_{κ}HC_{γ1} resulting in the vector pTRABacHuLC_{κ}V_{β-RMA}HC_{γ1}. TCR V_{β-RMA}HC_{γ1} chimeric proteins were produced in insect cells and purified as described supra. As an example of the use of such proteins (FIG. 9), C57/B16 mice were inoculated with 10,000 RMA T cell lymphoma cells and subsequently were treated 36 hours later with a composition comprising a RMA-specific V_{β}-Ig chimeric protein at a concentration of 500 μg/ml with the cytokine GM-CSF at a concentration of 100,000 IU/ml. This vaccination is given 1x+GM-CSF ×3 daily. The exact treatment protocol was repeated at 14-days intervals later. 60% of mice injected in this manner were alive at 40 days post tumor implant while more than 90% of control mice died of tumor challenge.

The sequence of the RMA Vb PCR Product is as follows (including the honey bee melittin signal sequence):

| | |
|---|---|
| ACTAGTTTTT ATGGTCGTGT ACATTTCTTA | |
| CATCTATGCG GACGCTGGAG TTACCCAGAC ACCCAGACAT | |
| GAGGTGGCAG AGAAAGGACA AACAATAATC CTGAAGTGTG | |
| AGCCAGTTTC AGGCCACAAT GACCTTTTCT GGTACAGACA GACCAAGATA | |
| CAGGGACTAG AGTTGCTGAG CTACTTCCGC AGCAAGTCTC TTATGGAAGA | |
| TGGTGGGGCT TTCAAGGATC GATTCAAAGC TGAGATGCTA AATTCATCCT | |
| TCTCCACTCT GAAGATTCAA CCTACAGAAC CCAAGGACTC AGCTGTGTAT | |
| CTGTGTGCCA GCAGTACCGG GACAGAAACG CTGTATTTTG GCTCAGGAAC | |
| CAGACTGACT GTTCTCGAGG ATCTGAAGGG CCC | (SEQ ID NO:58). |

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

1. A method for altering a T cell mediated pathology in a patient, said method comprising:
administering a composition comprising a chimeric protein;
said chimeric protein comprising at least a portion of a V_{β} or V₆₀ region of a TCR, and at least a portion of an immunoglobulin constant region;
wherein said V_{β} or V_{α} region is associated with a particular TCR from a T cell from said patient having said T cell mediated pathology; and
said administering of said composition alters said T cell mediated pathology in said patient.

2. The method of claim 1 wherein said composition further comprises a second chimeric protein comprising at least a portion of V_{α} or V_{β} region of a TCR, and at least a portion of a second immunoglobulin constant region.

3. The method of claim 1 wherein said immunoglobulin constant region comprises a human IgG_{γ1} constant region.

4. The method of claim 1 wherein said V_{α} or V_{β} region of a TCR of said chimeric protein is a V_{β}.

5. The method of claim 1 wherein said V_{α} or V_{β} region of a TCR of said chimeric protein is a V_{α}.

6. The method of claim 1 wherein said chimeric protein further comprises a linker region between said V_{α} or V_{β} region and said portion of an immunoglobulin constant region;
wherein said linker region is a portion of the C_{β} or C_{α} region of a TCR, but not the entire C_{β} or C_{α} region, or a synthetic linker region.

7. The method of claim 2 wherein said second chimeric protein further comprises a second linker region between said V_{α} or V_{β} region and said portion of an immunoglobulin constant region;
wherein said linker region is a portion of the C_{β} or C_{α} region of a TCR, but not the entire C_{β} or C_{α} region, or a synthetic linker region.

8. The method of claim 1 or 2 wherein said V_{α} or V_{β} region of a TCR of said first chimeric protein is a V_{β} and said V_{α} or V_{β} region of a TCR of said second chimeric protein is a V_{α}.

9. The method of claim 2 wherein said second immunoglobulin constant region comprises a human κ or λ constant region.

10. The method of claim 1 or 2 wherein said V_{β} region of a TCR is an entire V_{β} region.

11. The method of claim 1 or 2 wherein said V_{β} region comprises an entire V_{β} region and said portion of a C_{β} comprises the first nine amino acids from a TCR β chain constant region (C_{β}).

12. The method of claim 1 or 2 wherein said V_{α} region of a TCR is an entire V_{α} region.

13. The method of claim 6 or 7 wherein said V_{α} region comprises an entire V_{α} region and said linker region comprises the first nine amino acids from a TCR α chain constant region (C_{α}).

14. The method of claim 1 or 2 wherein said first or second immunoglobulin constant region is selected from the group consisting of a human IgG_{γ1} constant region, a human IgG_{γ2} constant region, a human IgG_{γ3} constant region, a human IgG_{γ4} constant region, a human IgA₁ constant region, a human IgA₂ constant region, a human IgM constant region, a human IgD constant region, a human IgE constant region, a human κ chain constant region, and a human λ chain constant region.

15. The method of claim 1 wherein said chimeric protein is produced by a method comprising:
isolating genes encoding said V_{β} or V_{α} regions of a TCR from T cells of said patient having said T cell mediated pathology;
inserting said genes encoding said V_{β} or V_{α} region of the TCR, a linker region, and the gene encoding said immunoglobulin constant region into an expression vector to allow the expression of said first chimeric protein;
producing said chimeric proteins by introducing said expression vector into insect cell lines; and
isolating said chimeric proteins.

16. The method of claim 15 further comprising the step of inserting a gene encoding either V_{β} or V_{α} region of the TCR, a linker region, and a gene encoding at least a portion of a second immunoglobulin constant region into said expression vector to allow the expression of said second chimeric protein.

17. The method of claim 15 or 16 wherein said linker region of said first or second chimeric protein is a portion of the C_{β} or C_{α} region of a TCR, but not the entire C_{β} or C_{α} region, or a synthetic linker region.

18. The method of claim 15 or 16 further comprising a step of conjugating said chimeric proteins to a carrier protein.

19. The method of claim 18 wherein said carrier protein is keyhole-limpet hemocyanin (KLH).

20. The method of claim 1 wherein said composition is further co-administered with a cytokine or chemokine.

21. The method of claim 20 wherein said cytokine is granulocyte-macrophage-colony stimulating factor (GM-CSF).

22. The method of claim 20 wherein said chemokine is a monocyte chemotactic protein 3 (MCR 3).

23. The method of claim 15 wherein said expression vector is a baculovirus expression vector.

24. The method of claim 23 wherein said baculovirus expression vector comprises a honey bee melittin secretory signal sequence and a human placental alkaline phosphatase secretory signal sequence.

25. The method of claim 24 wherein said baculovirus expression vector further comprises a baculovirus AcNPV p10 promotor and AcNPV polyhedrin promotor, said p10 promotor controls a honey bee melittin, and said polyhedrin promotor controls a human placental alkaline phosphatase.

26. The method of claim 25 wherein said genes encoding said V_{β} region of the TCR and said genes encoding said first immunoglobulin constant region are controlled by said p10 promotor in said baculovirus expression vector, said genes encoding said V_{α} region of the TCR and said genes encoding said second first immunoglobulin constant region are controlled by polyhedrin promotor in said baculovirus expression vector.

27. The method of claim 25 wherein said genes encoding said V_{β} or V_{α} region of the TCR, and said genes encoding said immunoglobulin constant region are controlled by either said p10 promotor or polyhedrin promotor in said baculovirus expression vector.

28. The method of claim 15 wherein said genes encoding said first immunoglobulin constant region comprises a human IgG_{γ1} gene.

29. The method of claim 16 wherein said second immunoglobulin constant region comprises a human κ or λ constant region gene.

30. The method of claim 15 or 16 wherein said gene encoding said immunoglobulin constant region is selected from the group consisting of a human IgG_{γ1} constant region, a human IgG_{γ2} constant region, a human IgG_{γ3} constant region, a human IgG_{γ4} constant region, a human IgA₁ constant region, a human IgA₂ constant region, a human IgM constant region, a human IgD constant region, a human IgE constant region, a human κ constant region and a human λ constant region.

31. The method of claim 15 wherein said first chimeric protein is TCR V_{β}-C_{β}-IgG_{γ1}, TCR V_{α}-C_{α}-κ or TCR V_{α}-λ.

32. The method of claim 16 wherein said first and second chimeric proteins are TCR V_{β}-C_{β}-IgG_{γ1} and TCR V_{α}-C_{α}-κ or TCR V₆₂ -C_{β}-IgG_{γ1} and TCR V_{α}-C_{α}-λ.

33. The method of claim 13 wherein said insect cell lines are Trichoplusia ni (Hi-5) or Spodoptera frugiperda (sf9) cell lines.

34. The method of claim 15 or 16 wherein said chimeric proteins are analyzed for expression by ELISA.

35. The method of claim 15 or 16 wherein said chimeric proteins are isolated using a protein selected from the group consisting of protein A, protein G, protein L and other proteins being able to bind to an immunoglobulin binding domain.

36. The method of claim 35 wherein said other protein able to bind an immunoglobulin binding domain is an anti-immunoglobulin antibody.

37. The method of claim 1 wherein said T cell mediated pathology is T cell lymphoma.

38. The method of claim 1 wherein said T cell mediated pathology is an autoimmune disease selected from the group consisting of multiple sclerosis, systemic lupus erythematosus, diabetes, inflammatory bowel disease, myasthenia gravis, rheumatoid arthritis, and thyroiditis.

39. A composition for altering a T cell mediated pathology in a patient comprising:
a chimeric protein, said chimeric protein comprising at least a portion of a V_{β} or V_{α} region of a TCR, and at least a portion of an immunoglobulin constant region, wherein said V_{β} or V_{α} region is associated with a particular TCR from a T cell clone from said patient having said T cell mediated pathology.

40. The composition of claim 39 wherein said chimeric protein further comprises a linker region between said V_{α} or V_{β} region and said portion of an immunoglobulin constant region;
wherein said linker region is a portion of the C_{β} or C_{α} region of a TCR, but not the entire C_{β} or C_{α} region, or a synthetic linker region.

41. A composition of claim 39 further comprising a second chimeric protein comprising at least a portion of a V_{β} and V₆₀ region of a TCR, and at least a portion of a second immunoglobulin constant region, wherein said V_{β} and V_{α} region are associated with a particular TCR from a T cell clone from said patient having said T cell mediated pathology.

42. The composition of claim 40 wherein said chimeric protein further comprises a linker region between said V_{α} or V_{β} region and said portion of an immunoglobulin constant region;
wherein said linker region is a portion of the C_{β} or C_{α} region of a TCR, but not the entire C_{β} or C_{α} region, or a synthetic linker region.

43. The composition of claim 39 or 40 wherein said chimeric proteins are produced in according to claim 13 or 14.

44. The composition of claim 39 or 40 wherein said immunoglobulin constant regions are selected from the group consisting of a human IgG_{γ1} constant region, a human IgG_{γ2} constant region, a human IgG_{γ3} constant region, a human IgG_{γ4} constant region, a human IgA₁ constant region, a human IgA₂ constant region, a human IgM constant region, a human IgD constant region, a human IgE constant region, a human κ chain constant region, and a human λ chain constant region.

45. The composition of claim 39 wherein said immunoglobulin constant region comprises IgG_{γ1} constant region operatively linked to said V_{β} region of the TCR.

46. The composition of claim 39 wherein said immunoglobulin constant region comprises κ or λ constant region operatively linked to said V_{α} region of the TCR.

47. The composition claim 40 wherein said two chimeric proteins are V_{β}-IgG_{γ1} and V_{α}-κ or V_{β}-IgG_{γ1} and V_{α}-λ.

48. The composition of claim 39 or 40 further comprises a carrier protein.

49. The composition of claim 48 wherein said carrier protein is keyhole-limpet hemocyanin (KLH).

50. The composition of claim 39 or 40 is further co-administered with a cytokine or chemokine.

51. The composition of claim 50 wherein said cytokine is granulocyte-macrophage-CSF.

52. The composition of claim 50 wherein said chemokine is a monocyte chemotactic protein 3 (MCP 3).

53. The composition of claim 39 or 40 wherein said composition comprises an immunotherapy.

54. The composition of claim 39 wherein said T cell mediated pathology is a T cell lymphoma.

55. The composition of claim 39 wherein said T cell mediated pathology is an autoimmune disease selected from the group consisting of multiple sclerosis, systemic lupus erythematosus, diabetes, inflammatory bowel disease, myasthenia gravis, rheumatoid arthritis, and thyroiditis.

56. The composition of claim 39 is further administered by injection, inhalation, oral or transdermal delivery.
